(19) United States
(12) Patent Application Publication
(10) Pub. No.: US 2024/0077439 A1
(43) Pub. Date: Mar. 7, 2024

(54) Methods and Systems for Measuring Citrate and Creatinine Levels by NMR Spectroscopy

(71) Applicant: Laboratory Corporation of America Holdings, Burlington, NC (US)

(72) Inventors: Steven P. Matyus, Burlington, NC (US); Erwin Garcia, Burlington, NC (US); Justyna E. Wolak-Dinsmore, Durham, NC (US)

(21) Appl. No.: 18/459,112

(22) Filed: Aug. 31, 2023

Related U.S. Application Data
(60) Provisional application No. 63/402,738, filed on Aug. 31, 2022.

(51) Int. Cl. G01N 24/08; G01N 33/487

(52) U.S. Cl. CPC G01N 24/08; G01N 33/487

(57) ABSTRACT

Methods and systems for detecting a presence and a concentration of biomarkers relevant to kidney stone formation may be useful in determining a personalized therapeutic approach for a subject. Nuclear Magnetic Resonance (NMR) spectroscopy may be a valuable tool in detecting various biomarkers related to various disease states. A biosample obtained from a subject may be examined using NMR spectroscopy to determine the presence and the concentration of relevant biomarkers.

### CROSS-REFERENCE TO RELATED APPLICATIONS

This application claims priority to U.S. Provisional Application No. 63/402,738, filed Aug. 31, 2022, which is incorporated by reference in its entirety herein.

### TECHNICAL FIELD

The present disclosure relates generally to methods and systems for determination of citrate and creatinine concentrations from an in vitro biological specimen, and, more particularly, determination of citrate and creatinine concentrations through the utilization of NMR spectroscopy.

### BACKGROUND

The prevalence of nephrolithiasis or kidney stones in the United States, based on a survey conducted from 2007-2014, was 10.1%, with the prevalence being higher in men (12.6%) than in women (7.5%). The prevalence of kidney stones has risen over the past few decades; an increase that coincides with an increase in obesity and type 2 diabetes. Identifying the causes of kidney stones as well as personalized treatment to reduce kidney stone formation and recurrence is thereby, a high priority.

### SUMMARY

Described herein are methods and systems that may aid in the identification and treatment of nephrolithiasis. Understanding kidney stone composition aligns with the high priority of identification and treatment of kidney stones. Kidney stones may comprise uric acid, cystine or struvite. Other markers that may identify patients at risk for kidney stone formation and recurrence are citrate and creatinine. Methods and systems for accurately and rapidly determining levels of citrate and creatinine may have useful implications for disease states such as nephrolithiasis and type 2 diabetes. The methods and systems described herein may accurately determine the amount of citrate and/or creatinine using nuclear magnetic resonance (NMR) spectroscopy.

The present disclosure may be embodied in a variety of ways. In some embodiments, methods and systems may include a determination of citrate and/or creatinine from a urine sample from a patient. In some embodiments, a method for detecting a presence of citrate and/or creatinine from urine may comprise the steps of acquiring a urine sample from a subject, acquiring an NMR spectrum of the urine sample obtained, and determining the concentration of citrate and/or creatinine or other metabolites of interest in the sample based on an NMR spectrum of the sample. In some embodiments, the method may include deconvolving the NMR spectrum and determining the concentration of citrate and/or creatinine or other metabolites of interest in the sample based on the deconvolved NMR spectrum of the sample. A concentration of the citrate and/or creatinine may be further calculated using a generated standard calibration curve. The calibration curve may be generated by relating the peak amplitude for urine concentrated with at least one of a creatinine and/or citrate standard to the amount of the added standard. In some embodiments, the determination of the concentration of citrate and/or creatinine in the sample may be used to determine a course of action for treatment for the subject. In some embodiments, the subject may be a patient undergoing treatment related to kidney stones.

In some embodiments, the method may include simultaneously detecting the presence of both citrate and creatinine based on their respective NMR signals. In some embodiments, the step of simultaneous detection may comprise the use of a mathematical deconvolution step. In some embodiments, various chemical analytes as described in more detail herein may be tested or added to the urine sample to examine the potential interference between the analyte NMR signal and the citrate and/or creatinine NMR signal. Thus, in some embodiments, the generation of citrate and/or creatinine test results may not be not hindered or interfered with by other analytes.

Some embodiments may be directed to systems comprising an NMR analyzer. The NMR analyzer may be configured to acquire a citrate and/or creatinine signal lineshape for a biosample. The NMR analyzer may include a computer program product that may store the measured citrate and/or creatinine lineshapes and reference spectra. The computer program may be configured to derive the concentrations of citrate and/or creatinine through a deconvolution process. The NMR analyzer may further include an NMR spectrometer, a flow probe in communication with the spectrometer, and/or a controller in communication with the spectrometer configured to obtain an NMR signal of a defined peak region of NMR spectra associated with citrate and creatinine in the flow probe. In some embodiments, the system may comprise a component to generate a patient report providing citrate and/or creatinine levels. The controller may include or be in communication with at least one local or remote processor, wherein the at least one processor may be configured to perform at least one of the following steps: (i) obtain a composite NMR spectrum of a fitting region of an in vitro biosample; (ii) deconvolve the composite NMR spectrum using a defined deconvolution model and curve fit functions; and/or (iii) mathematically calculate a concentration of citrate and creatinine from a generated calibration curve.

In some embodiments, a clinical analyzer, such as a Vantera® Clinical Analyzer, may be used for marker determination where the analyzer may be communicatively coupled to the NMR instrument for an automated, high throughput, reagent-less sampling process. In some examples, the clinical analyzer may be capable of autonomously mixing each urine sample with buffer on board such that sample preparation is automated once a sample is obtained. In some examples, a urine sample may be mixed with a buffer in a 2:1 (v/v) ratio.

Further features, advantages and details of the present disclosure will be appreciated by those of ordinary skill in the art from a reading of the figures and the detailed description of the embodiments that follow, such description being merely illustrative of the present disclosure. Features described with respect with one embodiment can be incorporated with other embodiments although not specifically discussed therewith. That is, it is noted that aspects of the disclosure described with respect to one embodiment, may be incorporated in a different embodiment although not specifically described relative thereto. That is, all embodiments and/or features of any embodiment can be combined in any way and/or combination. The foregoing and other aspects of the present disclosure are explained in detail in the specification set forth below.

### BRIEF DESCRIPTION OF THE DRAWINGS

The present disclosure may be better understood with reference to the accompanying drawings, in which embodiments of the disclosure may be shown. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art. Further, the flowcharts and block diagrams of certain of the figures herein illustrate the architecture, functionality, and operation of possible implementations of analysis models and evaluation systems and/or programs according to the present disclosure. In this regard, each block in the flow charts or block diagrams can represent a module, segment, operation, or portion of code, which may comprise one or more executable instructions for implementing the specified logical function(s). It should also be noted that in some alternative implementations, the functions noted in the blocks might occur in an order different than that noted in the figures. For example, two blocks shown in succession may in fact be executed substantially concurrently or the blocks may sometimes be executed in the reverse order, depending upon the functionality involved. The present disclosure may be better understood by referencing the following non-limiting figures.

FIG. 1 shows a flow diagram for a process of determining citrate and creatinine concentrations from a biosample in accordance with an embodiment of the present disclosure.

FIG. 2 shows a schematic representation of an NMR one-pulse, radio frequency pulse sequence experiment with WET solvent suppression in accordance with an embodiment of the present disclosure.

FIG. 3 shows a schematic representation of an NMR spectrum of a mixture of formate, creatinine, citrate, and TSP indicated in accordance with an embodiment of the present disclosure.

FIG. 4A shows a schematic representation of a single NMR peak of TSP for determination of peak center location in accordance with an embodiment of the present disclosure.

FIG. 4B shows a schematic representation of a single NMR peak of TSP for determination of peak baseline in accordance with an embodiment of the present disclosure.

FIG. 4C shows a schematic representation of a single NMR peak of TSP for determination of peak linewidth at 50% amplitude in accordance with an embodiment of the present disclosure.

FIG. 4D shows a schematic representation of a single NMR peak of TSP for determination of peak skew at 20% amplitude in accordance with an embodiment of the present disclosure.

FIG. 5A shows a schematic representation of a single NMR peak of formate for determination of peak center location in accordance with an embodiment of the present disclosure.

FIG. 5B shows a schematic representation of a single NMR peak of formate for determination of peak baseline in accordance with an embodiment of the present disclosure.

FIG. 5C shows a schematic representation of a single NMR peak of formate for determination of peak linewidth at 50% amplitude in accordance with an embodiment of the present disclosure.

FIG. 5D shows a schematic representation of a single NMR peak of formate for determination of peak skew at 10% amplitude in accordance with an embodiment of the present disclosure.

FIG. 6 shows a flow diagram for a routine to improve creatinine region fit by accounting for other peaks in a region in accordance with an embodiment of the present disclosure.

FIG. 7 shows a flow diagram for a routine to improve creatinine region fit by adjusting creatinine peak linewidth in accordance with an embodiment of the present disclosure.

FIG. 8A shows a schematic representation of an NMR spectrum representative of a citrate region in accordance with an embodiment of the present disclosure.

FIG. 8B shows a schematic representation of NMR spectra representative of citrate as a function of pH in accordance with an embodiment of the present disclosure.

FIG. 9 shows a flow diagram for lineshape deconvolution of citrate NMR peaks in accordance with an embodiment of the present disclosure.

FIG. 10 shows a schematic representation of NMR spectra representative of various concentrations of creatinine in accordance with an embodiment of the present disclosure.

FIG. 11 shows a schematic representation of NMR spectra representative of various concentrations of citrate in accordance with an embodiment of the present disclosure.

FIG. 12 shows a schematic of a block diagram of an NMR spectroscopy apparatus in accordance with an embodiment of the present disclosure.

FIG. 13 shows a schematic of a block diagram of a data processing system in accordance with an embodiment of the present disclosure.

FIG. 14 shows a plot displaying a limit of quantification for creatinine in accordance with an embodiment of the present disclosure.

FIG. 15 shows a plot displaying a limit of quantification for citrate in accordance with an embodiment of the present disclosure.

FIG. 16 shows two plots where a first plot (left) is a scatter plot of linearity of creatinine and a second plot (right) is a residual plot for creatinine in accordance with embodiments of the present disclosure.

FIG. 17 shows two plots where a first plot (left) is a scatter plot of linearity of citrate and a second plot (right) is a residual plot for citrate in accordance with embodiments of the present disclosure.

FIG. 18 shows various plots comparing the measurements of creatinine made by an NMR assay as compared to a chemistry assay in accordance with embodiments of the present disclosure.

FIG. 19 shows various plots comparing the measurements of citrate made by an NMR assay as compared to a chemistry assay in accordance with embodiments of the present disclosure.

FIG. 20 shows two calibration curves of citrate (left) and creatinine (right) used in conversion from amplitude to concentration in accordance with embodiments of the present disclosure.

### DETAILED DESCRIPTION

### Terms and Definitions

Like numbers refer to like elements throughout. In the figures, the thickness of certain lines, layers, components, elements or features may be exaggerated for clarity. Broken lines illustrate optional features or operations unless specified otherwise. The order of operations and/or steps illustrated in the figures or recited in the claims are not intended to be limited to the order presented unless stated otherwise.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “from about X to Y” mean “from about X to about Y.”

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer, or section. Thus, a first element, component, region, layer, or section discussed below could be termed a second element, component, region, layer or section without departing from the teachings of the present disclosure. The sequence of operations (or steps) is not limited to the order presented in the claims or figures unless specifically indicated otherwise.

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

“Sample” or “patient sample” or “biological sample” or “specimen” are used interchangeably herein. Non-limiting examples of liquid samples for analysis with the disclosed systems and methods can include blood or a blood product (e.g., serum, plasma, or the like), urine, nasal swabs, a liquid biopsy sample (e.g., for the detection of cancer), or combinations thereof. The term “blood” encompasses whole blood, blood product or any fraction of blood, such as serum, plasma, buffy coat, or the like as conventionally defined. Suitable samples include those which are capable of being deposited onto a substrate for collection and drying including, but not limited to: blood, plasma, serum, urine, saliva, tear, cerebrospinal fluid, organ, hair, muscle, or other tissue samples or other liquid aspirates.

The term “patient” or “subject” is used broadly and refers to an individual that provides a biosample for testing or analysis.

The term “clinical disease state” means an at-risk medical condition that may indicate medical intervention, dietary adjustments and/or regimens, therapy, therapy adjustment or exclusion of a certain therapy (e.g., pharmaceutical drug) and/or monitoring is appropriate. Identification of a likelihood of a clinical disease state can allow a clinician to treat, delay, or inhibit onset of the condition accordingly. The term Examples of clinical disease states include, but are not limited to, nephrolithiasis, CHD, CVD, stroke, type 2 diabetes, prediabetes, dementia, Alzheimer's, cancer, arthritis, rheumatoid arthritis (RA), kidney disease, pulmonary disease, COPD (chronic obstructive pulmonary disease), peripheral vascular disease, congestive heart failure, organ transplant response, and/or medical conditions associated with immune deficiency, abnormalities in biological functions in protein sorting, immune and receptor recognition, inflammation, pathogenicity, metastasis and other cellular processes.

The term “programmatically” means carried out using a computer program and/or software, processor or ASIC directed operations. The term “electronic” and derivatives thereof refer to automated or semi-automated operations carried out using devices with electrical circuits and/or modules rather than via mental steps and typically refers to operations that are carried out programmatically. The terms “automated” and “automatic” means that the operations can be carried out with minimal or no manual labor or input. The term “semi-automated” refers to allowing operators some input or activation, but the calculations and signal acquisition as well as the calculation of the concentrations of the ionized constituent(s) is done electronically, typically programmatically, without requiring manual input. The term “about” refers to +/−10% (mean or average) of a specified value or number.

The automated clinical NMR analyzer may be particularly suitable to analyze metabolites and/or lipoprotein data in in vitro blood serum and/or plasma samples or urine samples. The term “circuit” refers to an entirely software embodiment, or an embodiment combining software and hardware aspects, components, or features.

The term “protocol” refers to an automated electronic algorithm (typically a computer program) with mathematical computations, defined rules for data interrogation and analysis that manipulates NMR data to compensate for temperature sensitivity.

The term “computer network” includes one or more local area networks (LAN), wide area networks (WAN) and may, in certain embodiments, include a private intranet and/or the public Internet (also known as the World Wide Web or “the web”). The term “networked” system means that one or a plurality of local analyzers can communicate with at least one remote (local and/or offsite) control system. The remote-control system may be held in a local “clean” room that is separate from the NMR clinical analyzer and not subject to the same biohazard control requirements/concerns as the NMR clinical analyzer.

As used herein, the word “integral” with reference to an NMR spectrometer refers to an obtained NMR signal (spectrum) of a sample. The integral can refer to the area of a specific peak in the NMR spectrum. The area of the peak is proportional to the concentration of that particular species. Therefore, if a (constant) concentration standard is measured, the integral value will be relatively constant if the NMR spectrometer/instrument is performing correctly, e.g., the value is within a target range, such as +/−10%, and in some embodiments, +/− about 2%. Alternatively, the integral can be based on more than one peak, or even of all the peaks of the NMR spectrum, but it is more common to measure the area of one defined peak. The term “ppm” (parts-per-million) can be used to describe the position of one or more peak(s) on the x-axis of the NMR spectrum and corresponds to the energy or frequency of the radio waves that are absorbed.

The term “downfield” refers to a region/location on the NMR spectrum that pertains to the left of a certain peak/location/point (higher ppm scale relative to a reference). Conversely, the term “upfield” refers to a region/location on the NMR spectrum that pertains to the right of a certain peak/location/point.

When measuring a known concentration standard as a stand-alone “calibration” sample, the integral provides a good test for (day-to-day) performance that allows quantitative NMR without adding an internal standard to a respective biosample. A calibration sample may be an aqueous or a non-aqueous solution of a concentration standard that can be used to calculate a factor to normalize the integral produced by an instrument or set of instruments, in order for the instrument(s) to generate equivalent integral for a known amount of concentration standard in a given volume of sample.

The term “concentration standard” refers to a substance that is used to evaluate one or more peaks in an NMR spectrum. Examples of concentration standards include ethyl benzene solutions for organic systems (non-polar) and sodium acetate solutions for aqueous systems. In some embodiments, a TMA (trimethyl acetic acid) solution may be used as a concentration standard. The TMA solution can have a specific ionic strength so that it behaves as plasma/serum or other sample of interest would with respect to NMR behavior. Further, because the chemical shifts of Citrate and Creatinine May Vary with pH, TSP (3-(trimethylsilyl) propionic-2,2,3,3-d4 acid, sodium salt) and formate signals may be used as references to identify the signals of interest.

### Methods for Measuring Citrate and/or Creatinine

The present disclosure will now be described more fully hereinafter, in which embodiments of the disclosure are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

Described herein are novel methods including an assay for identifying risk of nephrolithiasis as obtained from a subject biosample that utilizes NMR technology that may be paired with an NMR analyzer, such as a Vantera® Clinical Analyzer for quick, high-throughput results. The novel assay circumvents an alternative spectrophotometrical-based assay for measuring citrate and creatinine. Traditionally, citrate and creatinine have been detected and quantified from a biosample through a time-consuming, chemical assay that may be prone to human-based erroneous results. Chemicals used for this traditional assay involve the enzymes citrate lyase and creatinase which are used for the detection of citrate and creatinine, respectively. Citrate may be quantified through a reduction-oxidation reaction that yields a change in absorbance of NADH while creatinine may be quantified by a change in absorbance as a result of reacting with alkaline picrate. Limiting features of this traditional assay include limited access to enzymes and/or enzyme suppliers, limited shelf life of the enzymes, and overly sensitive conditions for use of the enzymes. Thus, in the present disclsoure, a novel assay for the detection and quantification of citrate and creatinine using nuclear magnetic resonance is described.

In embodiments of the present disclosure, a biosample may be obtained from a subject and analyzed using analytical techniques to detect and quantify the presence of citrate and creatinine. An example of the embodiment may include a urine sample that can be analyzed using NMR technology.

In some embodiments, the analyte being measured may be citrate, where citrate can be found according to its respective signals in the NMR spectrum. In some embodiments, the analyte being measured may be creatinine, where creatinine can be found according to its respective signals in the NMR spectrum. Yet, in other embodiments of this disclosure, citrate and creatinine can be found simultaneously contemporaneously, using each analyte's respective signals in the NMR spectrum.

In some embodiments, a method may comprise: acquiring an NMR spectrum of a biosample obtained from a subject; and measuring a concentration of citrate and/or creatinine from the biosample based on the NMR spectrum. NMR analyzers may be particularly suitable to obtain data measurements of biosamples including qualitative and/or quantitative measurements that can be used for therapeutic or diagnostic purposes, and typically for diagnostic purposes that meet the appropriate regulatory guidelines for accuracy, depending on the jurisdiction and/or test being performed. In some embodiments, the auto-temperature compensation protocol will benefit the measurement of NMR quantifiable metabolites in human biofluids of the type of serum/plasma, urine, CSF, semen, sputum, lavages and the like.

In some embodiments, acquiring the NMR spectrum may comprise a step of producing a measured citrate and/or creatinine signal lineshape from the NMR spectrum. The acquiring of the NMR spectrum may further comprise generating a calculated lineshape for citrate and/or creatinine based on derived concentrations of citrate and/or creatinine expected to be present in the biosample.

In some embodiments, generating a calculated lineshape for citrate and/or creatinine may comprise calculating a plurality of reference coefficients for the calculated lineshape based on a linear least squares fit technique.

In some embodiments, discerning the concentration of citrate and/or creatinine may comprise determining a degree of correlation between an initial calculated lineshape and the measured citrate and/or creatinine signal lineshapes of the biosample. Further, a concentration or a presence of the analytes may be measured if the measured citrate and/or creatinine signal lineshape of the biosample is above a calculated threshold.

In some embodiments, the NMR spectrum of the biosample may include four citrate proton singlet signals in four distinct regions, wherein the four citrate proton singlet signals comprise a range of 2.50-2.75 ppm. Additionally, and/or alternatively in some embodiments, the NMR spectrum of the biosample may include two creatinine proton singlet signals in two distinct regions, wherein the two creatinine proton singlet signals comprise a range of 3.0-4.20 ppm. For example, in some embodiments, citrate and creatinine can be quantified using their respective NMR signals of 2.50-2.75 ppm and 3.07 ppm. The respective chemical shifts may vary in position according to pH. Thus, in certain embodiments, TSP (3-(trimethylsilyl) propionic-2,2,3,3-d4 acid, sodium salt) and/or formate signals may be used as relative references to identify the citrate and creatinine signals.

In some embodiments, the NMR spectrum of the sample can include a spectrum representative of citrate. Citrate may be identified by a singlet proton peak in four distinct regions of the NMR spectrum corresponding to four distinct chemical shifts. Particularly, the signal with the largest signal-to-noise ratio, citrate-3, may be identified first based on two relative distances: a first distance from formate and TSP and a second distance from the creatinine-2 signal and TSP. Citrate-4 may then be located based on its coupling with citrate-3. Citrate-2 may then be located based on its coupling with citrate-3. Citrate-1 may then be located based on its coupling with citrate-2. In some embodiments, using an expected amplitude for a given signal may improve citrate specificity when citrate signals are low relative to impurities, or in the presence of a close by and/or overlapping impurity peak(s).

In some embodiments, the NMR spectrum of the sample can include a spectrum representative of creatinine. Creatinine may be identified by a proton singlet in a first, downfield region and a proton singlet in a second, upfield region. Creatinine may be identified by the use of two signals, particularly, the upfield signal and its relative distance from the TSP standard. A mathematical equation may relate the distance from TSP to the upfield signal of creatinine as a function of pH. The two signals' amplitude may be used to limit ambiguity of identification. While both signals from creatinine may be used for quantification determination, the upfield signal may provide less interference with the water signal as result of high intensity.

In an embodiment, mathematical algorithms may be used to model a baseline accurately for citrate and creatinine signals. Thus, in some embodiments, the method may comprise deconvolving signal data associated with citrate and/or creatinine proton singlet signals. Further, the method may involve comparing data from a plurality of deconvolved signal data with previous calibration data corresponding to standard biosamples with known concentrations of citrate and/or creatinine to determine the concentration of citrate and/or creatinine in the biosample. Further, modelling mathematically may employ use of Lorentzian lineshapes, where a linear function and a constant offset may be incorporated in an algorithm. In some embodiments, a Lawson-Hanson non-negative least squares fitting algorithm may also be used for peak deconvolution.

In some embodiments, a unit conversion of signal amplitude to concentration of analyte in mg/dL or mg/L may comprise generating a linear calibration curve, plotting the signal amplitude of citrate or creatinine against the concentration used in preparing a respective solution. The calibration of curve may be performed in triplicate replicates with a total of at least 12 samples measured. With a linear plot, a mathematical function may be generated that can convert a given signal amplitude into units of concentration according to the calibration curve. Therefore, some embodiments may include a method producing a report which lists the concentrations of citrate and/or creatinine constituents present in the biosample.

In some embodiments, the NMR instrument may be couplable with a clinical analyzer, such as a Vantera® Clinical Analyzer, for automation of sampling. In such embodiments, a sample may be collected and deposited into the fully automated analyzer, where a spectrum may be provided shortly after. In some embodiments that do not use an automated sampling instrument, preparation of sample may comprise of generating a buffer and mixing the buffer with the sample prior to positioning within the NMR instrument. Yet, embodiments that utilize an automated sampling system, may only comprise of positioning sample within the instrument for sampling.

In some embodiments, each urine sample may be mixed within the Clinical Analyzer with a 2:1 (v/v) ratio of 1.5 molar potassium phosphate dibasic buffer further comprising 38 millimolar sodium formate and 2.18 millimolar TSP. The pH of the buffer may be acidic for examination. The pH may be adjusted with an acid to a pH of about 6.0. The sample may then be ready for spectral acquisition and processing.

In some embodiments, acquisition and processing may further comprise increasing temperature above room temperature within the probe, at least 4 steady-state scans, at least 2.0-second direct detection time, at least 1.95-second of relaxation time between scans, and at least a 64-second collection period. Further, a free-induction decay signal may be zero-filled with real and imaginary data points to be multiplied by an exponential window function that may correspond to a line-broadening of 0.5 Hz prior to Fourier transformation (FT). After FT, a spectrum may be redressed for phase and baseline errors. Other modifications to each of these variables may be used.

In an embodiment, analytical validation and imprecision may be examined by combining several urine pools selected from specimen samples and collected in a urine collection container. Three container samples of urine comprising a low, intermediate, and high citrate and creatinine concentrations may be evaluated for assay imprecision. An inter-assay and an intra-assay precision may be determined. Arithmetical mean, standard deviation, and percent coefficients of variation may be calculated, where acceptance criteria for citrate and creatinine assay imprecision may be predetermined to be 10% and 12%, respectively. Further, linearity may be evaluated using serial dilutions of the urine pools with respective low, intermediate, and high concentrations of citrate or creatinine. Linearity of assay may be assessed by linear and higher order polynomial regression of the assay results from the serially mixed pools compared to expected concentrations utilizing an EP Evaluator® software. Acceptance criteria for linearity data may be defined as allowable nonlinearity of 3.5% for citrate and 8.6% for creatinine, corresponding to a slope between 0.9-1.10. Table 1 displays intra- and inter-assay imprecision values for citrate and creatinine.

TABLE 1
Intra-/Inter-assay imprecision for citrate
and creatinine measured in urine.

| | Citrate (mg/L) | | | Creatinine (mg/dL) | | |
|---|---|---|---|---|---|---|
| | Low | Intermediate | High | Low | Intermediate | High |
| Intra-assay | | | | | | |
| (within-run)^{a} | | | | | | |
| Mean | 92.1 | 246.7 | 482.0 | 58.3 | 111.8 | 172.3 |
| SD | 2.7 | 2.2 | 4.2 | 0.4 | 0.5 | 1.2 |
| % CV | 2.9 | 0.9 | 0.9 | 0.7 | 0.4 | 0.7 |
| Inter-assay | | | | | | |
| (within-lab)^{b} | | | | | | |
| Mean | 90.3 | 245.8 | 478.0 | 58.1 | 112.4 | 173.0 |
| SD | 3.3 | 7.7 | 13.9 | 0.9 | 2.2 | 3.6 |
| % CV | 3.7 | 3.2 | 2.9 | 1.5 | 2.0 | 2.1 |

^{a}Based on 1 run of 20 tests (n = 20)
^{b}Based on CLSI EP5-A2 tested using 2 runs per day in duplicate for 20 days (n = 80).

In another embodiment, a limit of blank may be calculated using five deionized water samples. Further, a limit of detection may be calculated using five low-citrate and creatinine concentrations samples.

Further, temperature stability of citrate and creatinine from a sample may be examined. A room-temperature (20-25° C.), a refrigerated (2-8° C.), and a frozen (−20<−70° C.) assay may be utilized to examine effects from various temperatures including effects from freeze-thaw cycles of urine samples. Table 2 displays a summary of citrate and creatinine time-based/temperature-based stability.

TABLE 2
Summary of citrate and creatinine stability in urine specimens.

| Storage Condition | Citrate | Creatinine |
|---|---|---|
| Controlled Room Temp., | Up to 29 days | Up to 29 days |
| 20 to 25° C. | | |
| Refrigerated, 2 to 8° C. | Up to 29 days | Up to 29 days |
| Frozen, < −70° C. | Up to 49 days | Up to 49 days |
| Freeze-Thaw, −70° C. | 5x | 5x |

In one embodiment of the present disclosure, a results comparison of chemical-based assays versus NMR-based assays for citrate and creatinine concentrations may be performed. The results comparison examination may compare method results using the NMR-based assay compared to the citrate lyase and creatinase-based enzymatic assays traditionally used. Assay results may be compared to evaluate accuracy of the novel NMR-based assay for citrate and creatinine concentrations.

An embodiment may further examine interference of other analytes with the concentrations and results of citrate and creatinine from the NMR-based assay. At least 10 or more substances may be used for examination of interference wherein 3 of the 10 substances are endogenous substances and 7 of the 10 substances are exogenous substances. In one example of the current disclosure, the 3 endogenous substances may comprise, as an example, urea, uric acid, and albumin. The 7 exogenous substances may comprise, as an example, acetaminophen, acetic acid, acetylsalicylic acid, ascorbic acid or vitamin C, boric acid, ibuprofen and naproxen sodium. Boric or acetic acid may be used in place of hydrochloric acid as a preservative in urine samples. Substance concentrations, when compared to urine citrate and creatinine concentrations, may be examined at endogenous (naturally occurring) concentrations and exogenous (external and/or therapeutic) concentrations and higher.

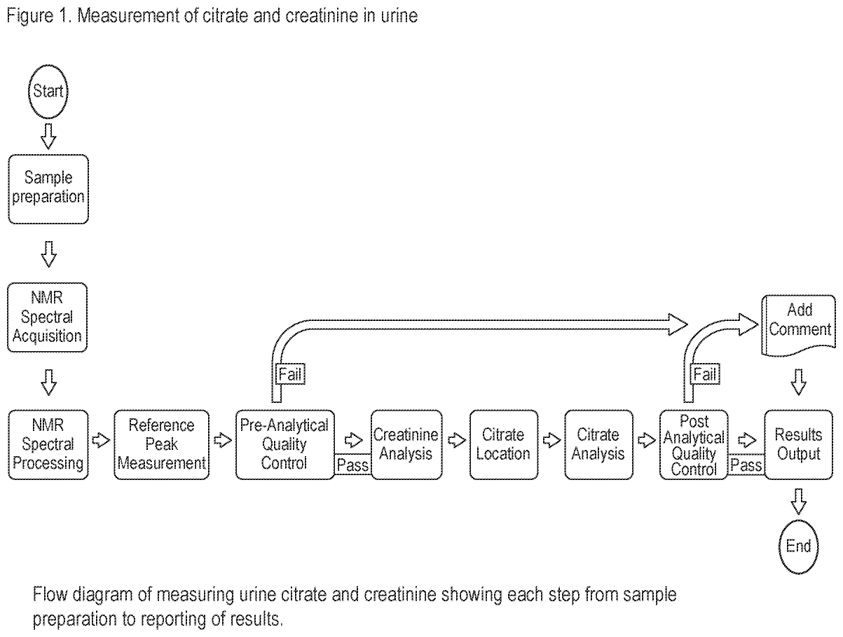

FIG. 1 shows a flow diagram for a process of determining citrate and creatinine concentrations from a biosample. A biosample may first be obtained from a subject in a biosample container. The biosample may then be placed on board the analyzer, such as a Vantera® analyzer. The analyzer may then prepare the biosample according to the following sample preparation procedure:

In some embodiments, there may be defined dilutions of the biological sample. For example, in some embodiments, each urine sample for the urine citrate and creatinine (UCC) assay can be mixed with buffer in a 2:1 (v/v) ratio on board on an analyzer such as a Vantera® Clinical Analyzer. The buffer may be comprised of 1.5 M potassium phosphate dibasic (K₂HPO₄; Sigma-Aldrich), 38 mM sodium formate (CHNaO₂, Sigma-Aldrich) and 2.18 mM 3-(trimethylsilyl)propionic-2,2,3,3-d4 acid, sodium salt (TSP; Sigma-Aldrich). The pH of the buffer can be adjusted to 6.0+0.1 with 6N HCl. The prepared sample can be delivered by the analyzer to the NMR flowcell for spectral acquisition and processing.

Sill referring to FIG. 1, following sample preparation, NMR spectral acquisition and spectral processing may follow. A one-dimensional, proton NMR spectrum can be collected using a one-pulse sequence. A 90° flip angle can be used as read pulse and a total acquisition time of 64 s. Other acquisition parameters may be as follows: spectral width=4496.4 Hz, steady state scans=4, direct detection time=2.0 s, relaxation between scans=1.95 s, number of scans=12. The free-induction decay signal can be zero-filled to 16,384 pairs of real and imaginary data points and can be multiplied by an exponential window function corresponding to a line-broadening of 0.5 Hz prior to Fourier transformation (FT). After FT, the spectrum can be corrected for phase and baseline errors.

Reference peak measurement may follow spectral acquisition and spectra processing. Characteristics of the TSP, formate, and creatinine peaks can be used for pre-analytical quality control, as chemical shift references, and inputs to the algorithm for quantification of citrate and creatinine. The peak center location, amplitude, linewidth at 50% amplitude and skew at 20% and 10% may be calculated for TSP and formate, respectively, while the peak center location of the two creatinine peaks at approximately 3.07 ppm and 4.12 ppm may be calculated.

At this point, pre-analytical quality control can be used to detect instrument failure modes which can ensure the urine citrate and creatinine assay is not performed unless the input spectra have been properly acquired under specified conditions (FIG. 1). Failure modes detected can include sample delivery failure and NMR shimming failure. If a given condition can be found to be present, subsequent evaluations may not be performed. This provides specificity to the reason for pre-analytical QC failure. Pre-analytical QC evaluations may be performed in the order presented below. Or other sequences of these steps may be used.

The software may detect complete sample delivery failure if the TSP amplitude is <4.5 au for any of the input spectra.

The software may detect partial sample delivery failure if TSP skew is <−1.0 data points or TSP skew is >3.0 data points for any of the input spectra.

The software may detect shimming failure if the TSP line width is >2.0 Hz and the formate line width is >2.0 Hz for any of the input spectra.

The NMR spectrum of the sample can include a spectrum representative of creatinine. Still referring to FIG. 1, creatinine may be identified by a proton singlet in a first, downfield region and a proton singlet in a second, upfield region. Creatinine may be identified by the use of two signals, particularly, the upfield signal and its relative distance from the TSP standard. A mathematical equation may relate the distance from TSP to the upfield signal of creatinine as a function of pH. The two signals' amplitude may be used to limit ambiguity of identification. While both signals from creatinine may be used for quantification determination, the upfield signal may provide less interference with the water signal as result of high intensity.

In some embodiments, citrate location can be identified following creatinine analysis (FIG. 1). As mentioned previously, in some embodiments, the NMR spectrum of the sample can include a spectrum representative of citrate. Citrate may be identified by a singlet proton peak in four distinct regions of the NMR spectrum corresponding to four distinct chemical shifts. Particularly, the signal with the largest signal-to-noise ratio, citrate-3, may be identified first based on two relative distances: a first distance from formate and TSP and a second distance from the creatinine-2 signal and TSP. Citrate-4 may then be located based on its coupling with citrate-3. Citrate-2 may then be located based on its coupling with citrate-3. Citrate-1 may then be located based on its coupling with citrate-2. In some embodiments, using an expected amplitude for a given signal may improve citrate specificity when citrate signals are low relative to impurities, or in the presence of a close by and/or overlapping impurity peak(s). In some embodiments, the analysis of citrate is followed by a post-analytical quality control as described herein (FIG. 1). Results of analysis may then be outputted using mathematical models and lineshape functions to convert amplitude of citrate and creatinine peaks into concentration units, such as for example, mg/L or mg/dL, respectively, for example.

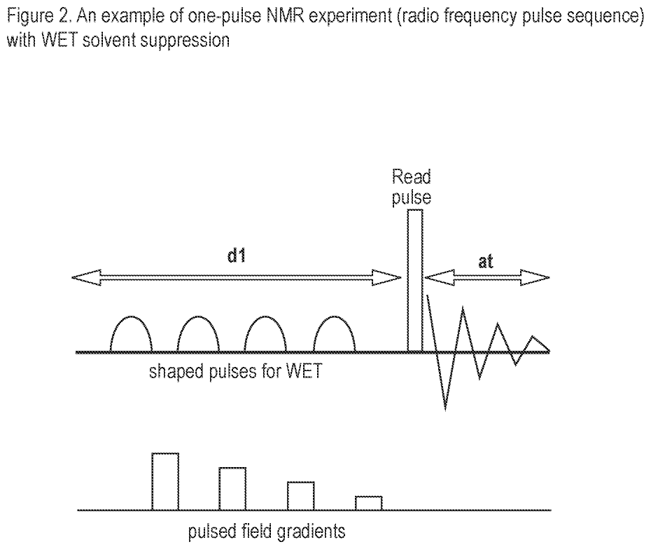

FIG. 2 shows a schematic representation of an NMR one-pulse, radio frequency pulse sequence experiment with WET solvent suppression. A proton NMR spectrum can be collected using a one-pulse sequence at 47° C. The solvent signal may be attenuated with water suppression enhanced through T1 effects (WET) module applied for 68 milliseconds.

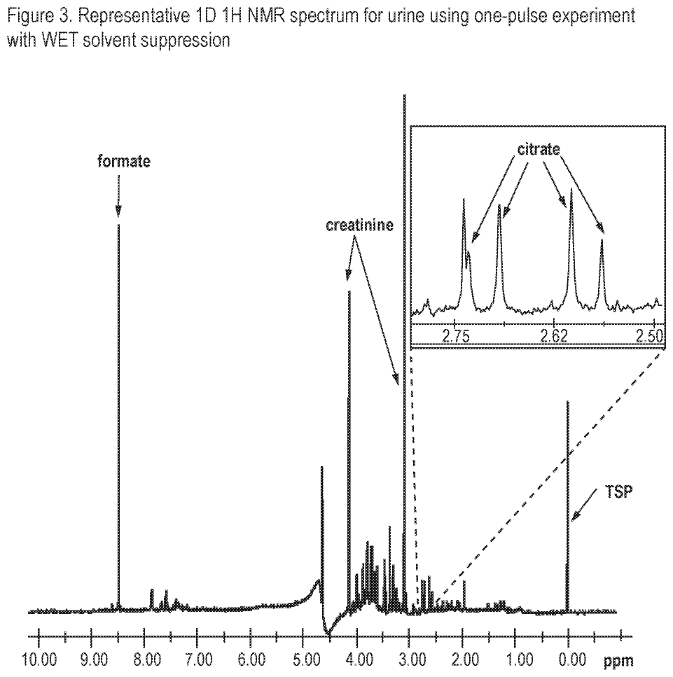

FIG. 3 shows a schematic representation of an NMR spectrum of a mixture of formate, creatinine, citrate, and TSP indicated. Inlayed in the spectrum is a zoomed-in schematic of the four citrate singlet peaks ranging in location from 2.50 ppm to 2.75 ppm. The TSP peak can be seen at 0.0 ppm and can provide as an internal standard for detecting creatinine and citrate peaks. The formate peak can also be noted at a range of 8.00 ppm to 9.00 ppm and can further provide as an internal standard for detecting creatinine and citrate. Peaks noted other than creatinine, citrate, TSP, and formate, may be considered irrelevant to the detection and quantification of creatinine and citrate. Other peaks identified may include analytes that may be found in the biosample, particularly urine. Further, other peaks identified may include impurities.

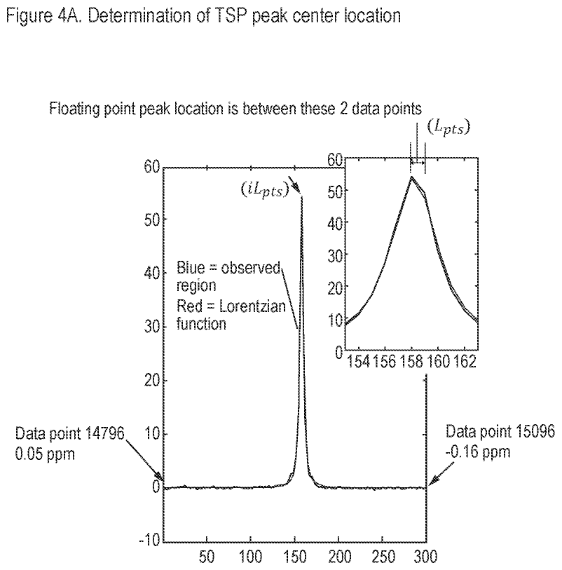

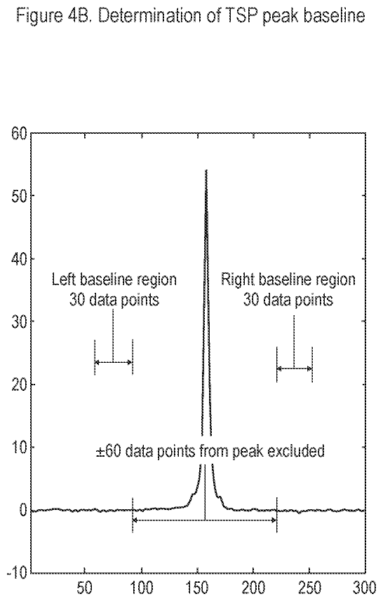

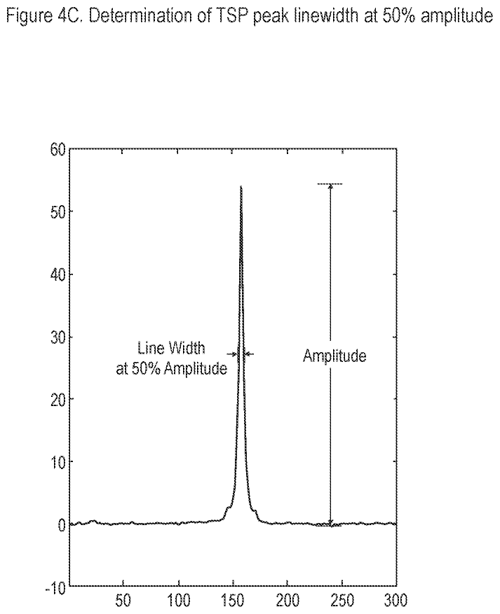

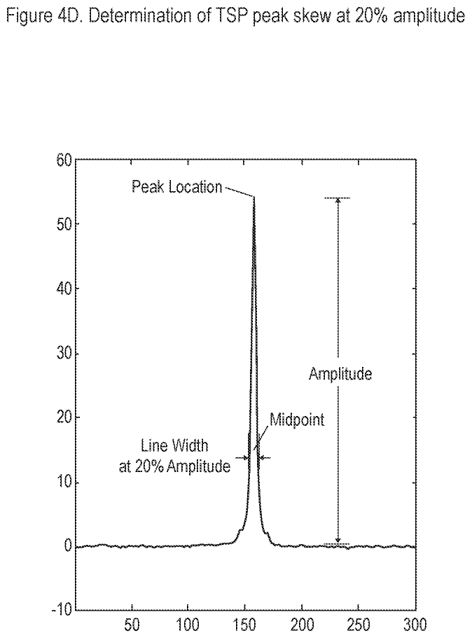

FIGS. 4A-4D show a schematic representation of a single NMR peak of TSP for determination of peak center location, peak baseline, peak linewidth at 50% amplitude, and peak skew at 20% amplitude, respectively. The x-axis displays data point location, and the y-axis displays peak amplitude. The peak center location (iLₚₜₛ) of the TSP peak can be determined as the highest value data point in the interval [14796,15096]. Using the derivative of the signal amplitude in the data point interval, this location could coincide with a change in sign of the derivative in order to ensure it is an actual peak, The floating point peak center location (Lₚₜₛ) can be determined by calculating the root mean square deviation (RMSD) between the observed TSP peak and a Lorentzian function centered in 0.01 data point increments around the integer peak center locations. The location for the Lorentzian function with the minimum RMSD can be the TSP floating point peak center location.

In some embodiments, in order to calculate the TSP linewidth at 50% peak amplitude, the peak baseline and amplitude can be first determined. A baseline for the TSP peak can be determined as the mean amplitude of the left and right baseline. The left and right baselines can be determined as median data point (value) in the intervals [iLₚₜₛ−90, iLₚₜₛ−60] and [iLₚₜₛ+60, iLₚₜₛ+90], respectively. 60 data points around the integer location can be excluded from consideration of the baseline because those points constitute (approximately) the TSP peak itself. TSP amplitude can be determined as the amplitude value at the integer peak center location data point minus the value of the baseline. TSP line width can be calculated as the length of a line drawn at 50% of the peak amplitude. Linear interpolation may be used to determine the intersection of the 50% line with the left and right sides of the peak. If no intersection at 50% exists on either the left or right side, the line may be assumed to extend to end of the interval [14796,15096] as appropriate.

TSP skew can be calculated as a difference between the floating point peak center location and the midpoint of a line drawn at 20% of the peak amplitude. The TSP peak can have a lineshape which includes satellite peaks on either side of the center peak and 20% height can be above the amplitude of these satellite peaks such that they do not contribute to error in measurement of skew. As with line width, linear interpolation is used to determine the intersection of the 20% line with the left and right sides of the peak. If no intersection at 20% exists on either the left or right side, the line can be assumed to extend to end of the interval [14796,15096] as appropriate.

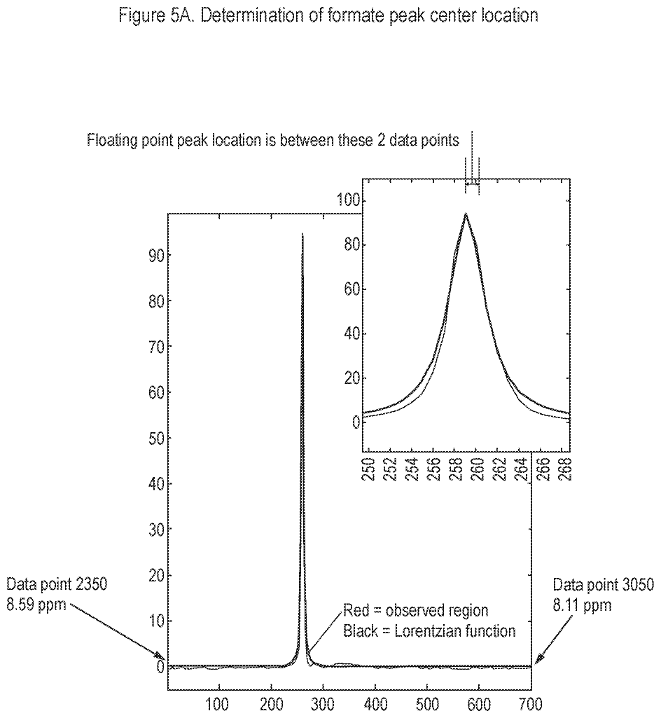

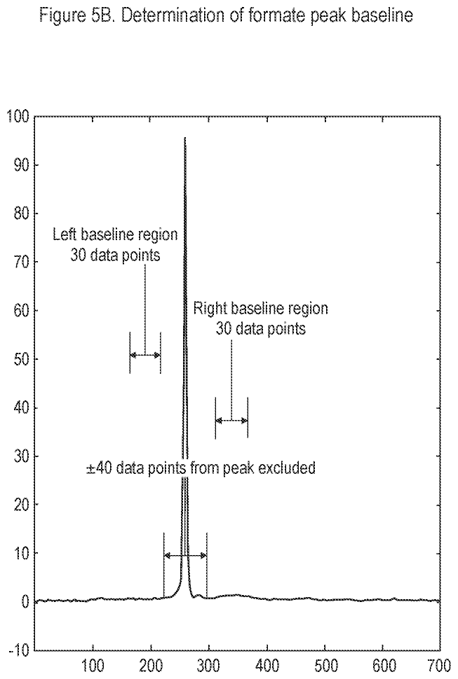

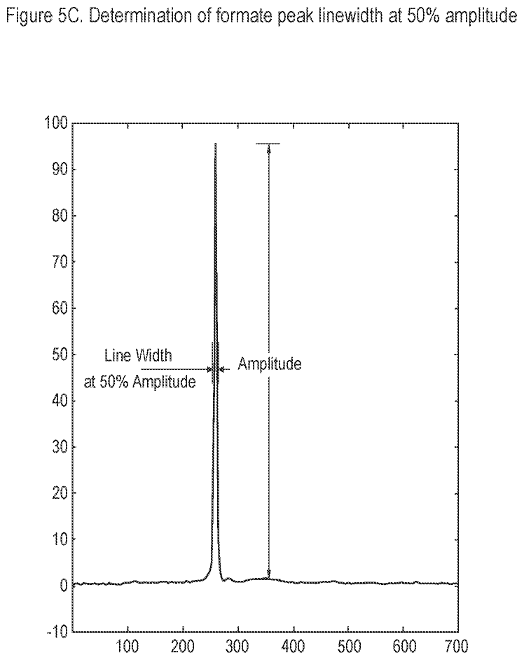

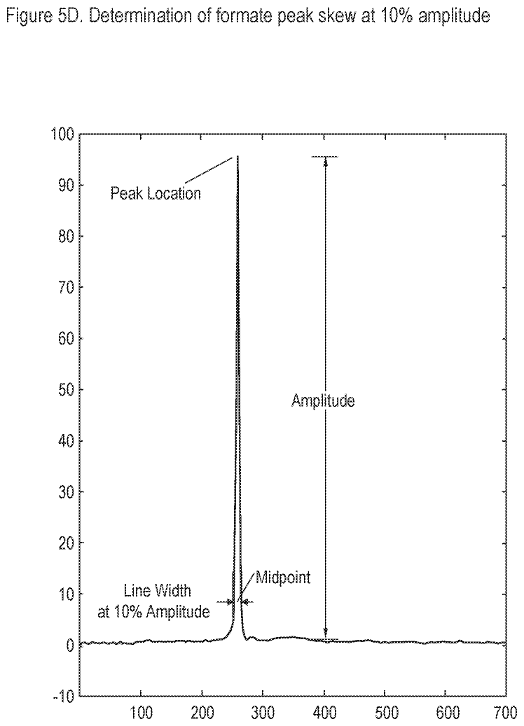

FIGS. 5A-5D show a schematic representation of a single NMR peak of formate for determination of peak center location, peak baseline, peak linewidth at 50% amplitude, and peak skew at 10% amplitude, respectively. The x-axis displays data point location, and the y-axis displays peak amplitude. The integer peak center location (iLₚₜₛ) of the formate peak can be determined as the highest amplitude value in the data point interval [2350,3050]. Using the derivative of the signal amplitude in the data point interval, this location could coincide with a change in sign of the derivative in order to ensure it is an actual peak. The floating point peak center location (Lₚₜₛ) can be determined by calculating the root mean square deviation (RMSD) between the observed formate peak and a Lorentzian function centered in 0.01 data point increments around the integer peak center location. The location of the Lorentzian function with the minimum RMSD can be the Formate floating point peak center location.

In some embodiments, in order to calculate the formate linewidth at 50% peak amplitude, the peak baseline and amplitude can be first determined. A baseline for the formate peak can be determined as the mean amplitude of the left and right baseline. The left and right baselines can be determined as median amplitude (value) at the intervals [iLₚₜₛ−70, iLₚₜₛ−40] and [iLₚₜₛ+40, iLₚₜₛ+70], respectively. 40 data points around the integer location can be excluded from consideration of the baseline because those points constitute (approximately) the formate peak itself. Formate amplitude can be determined as the value of the integer peak center location data point minus the value of the baseline. Formate line width can be calculated as the length of a line drawn at 50% of the peak amplitude. Linear interpolation can be used to determine the intersection of the 50% line with the left and right sides of the peak. If no intersection at 50% exists on either the left or right side, the line can be assumed to extend to end of the interval [2350,3050] as appropriate.

Formate skew can be calculated as a difference between the floating point peak center location and the midpoint of a line drawn at 10% of the peak amplitude. As with line width, linear interpolation can be used to determine the intersection of the 10% line with the left and right sides of the peak. If no intersection at 10% exists on either the left or right side, the line can be assumed to extend to end of the interval [2350,3050] as appropriate

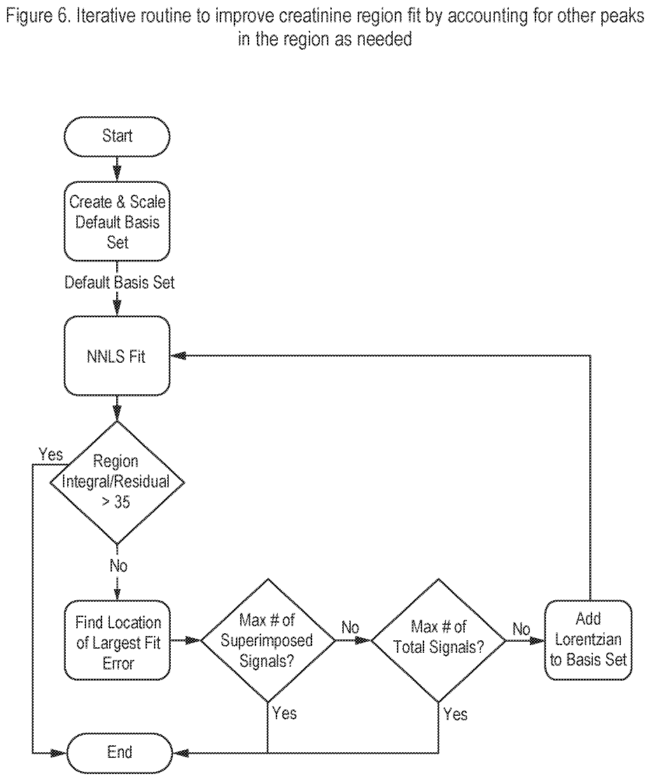

FIG. 6 shows a flow diagram for a routine to improve creatinine region fit by accounting for other peaks in a region. After acquisition, a basis set or design matrix may be generated as a sum of four Lorentzian lineshapes centered on a floating point of the analyte's respective location on peaks.

An embodiment of the method for analyzing creatinine described herein may be applied to the upfield peak only but analysis of each peak may produce a concentration value for creatinine. Thus, still referring to FIG. 6, an iterative method using a Lawson-Hanson non-negative least squares deconvolution can be used to fit each creatinine peak using a basis set (design matrix). The creatinine basis set can include 5 Lorentzian singlets centered on the floating point creatinine location, 2 sloped lines, and a DC offset. The lineshape of the Lorentzian singlets can be divided into left and right linewidths. The default left and right linewidths may be half of the measured creatinine line width. The area of the Lorentzian lineshapes may be normalized to a value of 1000.

As further shown in FIG. 6, the fit deviation resulting from each application of the deconvolution method may be evaluated to determine the location of an additional Lorentzian component. These additional components can improve the accuracy of the fit by modeling additional or neighboring signals in the fitting region which may be highly variable sample to sample.

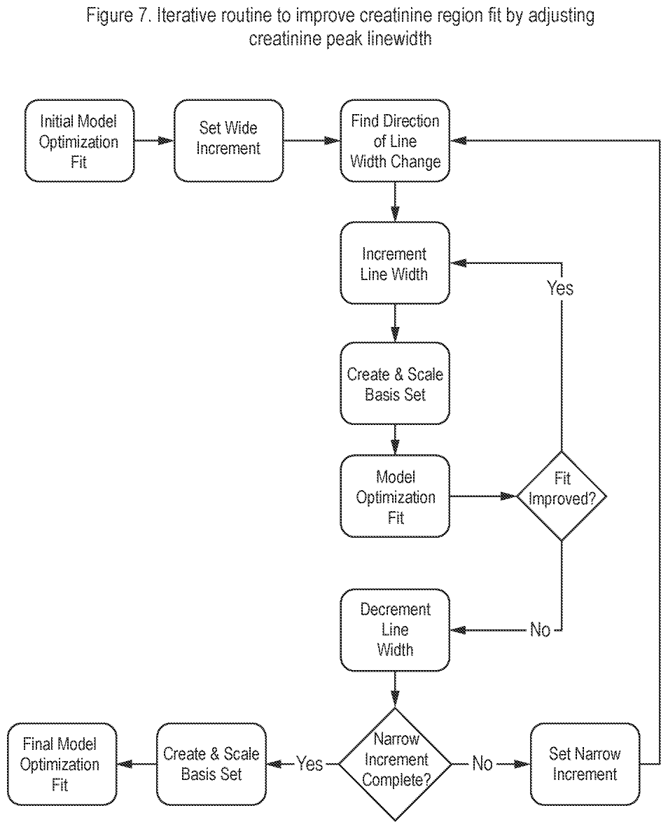

FIG. 7 shows a flow diagram for a routine to improve creatinine region fit by adjusting creatinine peak linewidth. In addition to iterating the fit to resolve neighboring signals in the fitting region, the fit can be iterated to alter the linewidth of the Lorentzian lineshapes used to fit creatinine and the neighboring signals. Ultimately, final lineshape attributes of the upfield creatinine peak (left and right linewidths) may be utilized in the downstream citrate modeling routine.

In some embodiments, once the creatinine fit is optimized, the creatinine concentration (e.g., mg/dL) can be calculated. This may be done using the following analysis or a variation thereof. Thus, in an embodiment, the creatinine concentration may be calculated by summing the deconvolution coefficients of the creatinine Lorentzian components and multiplying by (8836.9*0.011312) for the upfield creatinine peak. The number 8836.9 can convert the value to μmol/L and the number 0.011312 can convert the μmol/L value to mg/dL.

The distance (difference) between the TSP and formate locations may be an indication of pH and can be useful for calculation of other pH-dependent peak locations and peak couplings (the distance between two peaks). The predicted location of the upfield creatinine peak (referred to as CRE1 hereafter) as well as the predicted coupling between the downfield and upfield creatinine peaks can be calculated from the TSP and formate locations per the formula provided herein according to examples of the present disclosure.

The predicted coupling between the two creatinine peaks can be calculated as:

CRE_coupling=(a*exp(b*NMRpH)+c*exp(d*NMRpH))+1350

where: a=−0.06187; b=0.02749; c=349.9; d=−0.0002149;

NMRpH=tsp·fPosition−formate·fPosition−12050.

The predicted location of the upfield creatinine peak can be calculated as:

CRE1_location=tsp·fPosition−((a*exp(b*NMRpH)+c*exp(d*NMRpH))+4400)

where: a=−0.02564; b=0.02853; c=182.3; d=−0.0002739;

NMRpH=tsp·fPosition−formate·fPosition−12050.

The predicted location of the downfield creatinine peak (referred to as CRE2 hereafter) can be calculated in two steps. First, the predicted location can be calculated from the floating point TSP location and NMR-pH:

CRE2_location=tsp·fPosition−((a*exp(b*NMRpH)+c*exp(d*NMRpH))+5900)

where: a=−0.07221; b=0.02848; c=382.4; d=−0.0003509;

NMRpH=tsp·fPosition−formate·fPosition−12050.

Second, it can be independently calculated from the predicted upfield creatinine location and coupling:

CRE2_location=CRE1_location−CRE_coupling

In some embodiments, the predicted location of CRE2 can be assigned as the most downfield result (whichever result is the left-most or smaller data point value). The predicted CRE2 location can be used to define the creatinine doublet search region. This search region can be defined as the predicted CRE2 location—100 data points to the predicted CRE1 location +40 data points. Within this search region, pairs of peak matching the predicted coupling ±50 data points may be identified. An empirically derived cost function can then be used to choose the pair of peaks of highest amplitude most closely matching the known amplitude relationship of the creatinine peaks as well as their linear, pH-dependent coupling relationship with TSP. The creatinine doublet search region can be smoothed using a Savitzky-Golay filter of polynomial order 3 and frame length 11. The derivative of the smoothed search region can be calculated. Peaks can occur where the derivative changes sign from positive to negative. Pairs of peaks separated by the predicted coupling ±50 data points can be identified.

The pair of peaks which can maximize the following function can be chosen as the creatinine doublet (CRE2 and CRE1 peaks):

((lamp+ramp)/2)/(abs(0.6−lamp/ramp)*(abs(2.7629*(tsp_loc−rloc)−6379.2−(tsp_loc−lloc)))+1)
where: lamp=estimated amplitude of left (downfield, CRE2) peak;
ramp=estimated amplitude of right (upfield, CRE1) peak;
lloc=location of left (downfield, CRE2) peak;
rloc=location of right (upfield, CRE1) peak;
tsp_loc=location of TSP peak.

Floating point locations for the individual creatinine peaks (CRE2 and CRE1) can be determined by calculating the root mean square deviation (RMSD) between the observed creatinine peak and a Lorentzian function centered in 0.01 data point increments around the integer peak location. Similar to TSP and formate, a baseline for each creatinine peak can be determined as the mean amplitude of the left and right baseline.

For the upfield creatinine peak (CRE1), the left and right baselines can be determined as a median amplitude (value) in the intervals [iLₚₜₛ−80, iLₚₜₛ−70] and [iLₚₜₛ+70, iLₚₜₛ+80], respectively, where iLₚₜₛ refers to the integer peak location. 70 data points around the peak location may be excluded from consideration of the baseline because those points constitute (roughly) the peak itself.

For the downfield creatinine peak (CRE2), the left and right baselines can be determined as median amplitude (value) at the intervals [iLₚₜₛ−20, iLₚₜₛ−15] and [iLₚₜₛ+15, iLₚₜₛ+20], respectively, where iLₚₜₛ refers to the integer peak location. 15 data points around the peak location may be excluded from consideration of the baseline because those points constitute (roughly) the peak itself. Baseline regions for the downfield creatinine peak may be smaller because of its proximity to the residual water peak which may distort the baseline. The amplitude of each creatinine peak can be determined as the amplitude value at the integer peak location minus the value of its baseline.

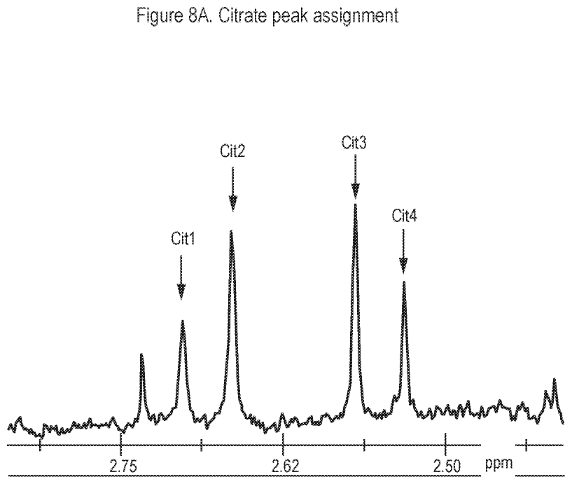

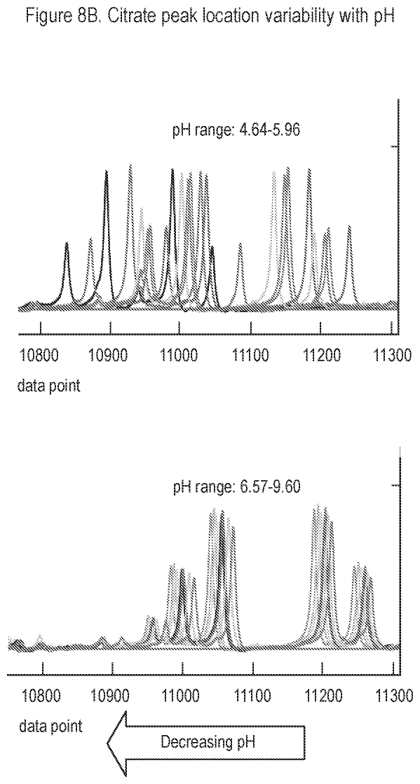

FIGS. 8A and 8B shows a schematic representation of an NMR spectrum representative of citrate and a dependence of location on pH, respectively. A location of citrate peaks may be very sensitive to pH. Coupling of peaks 1 and 2 (most downfield, labeled in FIG. 8A as cit1 and cit2) and peaks 3 and 4 (most upfield, labeled in FIG. 8A as Cit3 and Cit4) may be relatively constant with pH. Coupling between the two pairs, however, may be sensitive to pH. Citrate may be located utilizing peak locations of TSP and formate as an indicator of pH, as well as the locations determined for the two creatinine peaks as a second indicator of pH.

In some embodiments, the upfield doublet may be identified first because their position can be less pH-sensitive than the downfield doublet. Given the location of the upfield doublet, the pH-based coupling can used to identify the downfield doublet. The floating-point location of the 4 citrate peaks may be used as inputs to the method for modeling the citrate region.

The predicted Cit3 peak location may be first calculated based on the observed difference between the formate and TSP locations, and second based on the observed difference between the downfield creatinine peak and TSP locations. The final predicted Cit3 peak location may be calculated as the mean of the two separate estimates of the Cit3 location.

For example, in an embodiment, the predicted Cit3 peak location based on TSP and formate may be calculated as follows:

Citr3_formate_location=tsp·fPosition−((a0+a1*cos(NMRpH*w)+b1*sin(NMRpH*w)+a2*cos(2*NMRpH*w)+b2*sin(2*NMRpH*w)+a3*cos(3*NMRpH*w)+b3*sin(3*NMRpH*w))+3700)

where: NMRpH=tsp·fPosition−formate·fPosition−12050;

a0=−805.5; a1=1407; b1=1258; a2=59.14; b2=−868.8; a3=−157;
b3=94.78; w=0.00763.
The predicted Cit3 peak location based on TSP and CRE2 may be calculated as follows:

Citr3_cre2_location=tsp·fPosition−(((p1*NMRpH\hat{ }2+p2*NMRpH+p3)/(NMRpH\hat{ }3+q1*NMRpH\hat{ }2+q2*NMRpH+q3))+3700)

Where: NMRpH=tsp·fPosition−cre2·fPosition−5900;p1=−1.599e+09;

p2=7.1e+11; p3=−5.406e+12; q1=−2.574e+06; q2=−4.598e+08;
q3=6.171e+11

If the NMR-pH in the equation based on CRE2 above is >380, the Cit3 location based on CRE2 may be skipped. The predicted location of the third citrate peak (Cit3) can be calculated as an average of predicted locations based on formate and CRE2:

Citr3_location=(Citr3_formate_location+Citr3_cre2_location)/2

If the CRE2 based location is skipped,

Citr3_location=Citr3_formate_location

In certain embodiments, the predicted Cit3 location can then be used to define the upfield citrate doublet search region. This search region can be defined as 40 data points downfield from the predicted Cit3 location to 97 data points upfield from the predicted Cit3 location. Within this search region, pairs of peaks matching the known coupling of the Cit3 and Cit4 peaks may be identified. An empirically derived cost function may then be used to choose the pair of peaks of highest amplitude most closely matching the known amplitude relationship of the Cit3 and Cit4 peaks. The upfield citrate doublet search region may then be smoothed using a Savitzky-Golay filter of polynomial order 3 and frame length 9.

In one embodiment, the derivative of the smooth search region can be calculated. Peaks occur where the derivative changes sign from positive to negative. For each peak identified, the estimated amplitude of the peak can be calculated by summing the like-signed values of the derivative on either side of the peak location and dividing the sum by 2.

In one embodiment, for each peak identified, if a peak paired by 57+6 data points is not already identified as a peak by derivative sign change, the amplitude of the search region paired by 57 data points can be added as “virtual” peak with amplitude calculated as a one-sided sum of like-signed derivate values at the paired point. The addition of “virtual” peaks allows for the possibility of the doublet to be identified even when obscured by other non-citrate peaks. Pairs of peaks (actual and virtual) separated by 57±6 data points may be identified. The pair of peaks which can maximize the following function are chosen as the upfield citrate doublet (Cit3 and Cit4 peaks):

(((lamp+ramp)/2)\hat{ }2)/abs(36.8/22.13−lamp/ramp)

where: lamp=estimated amplitude of left (downfield, Cit3) peak;
ramp=estimated amplitude of right (upfield, Cit4) peak.

This empirically derived function can effectively weigh the amplitude relationship of the peak pairs to prefer peak pairs which can match the typical amplitude ratio of the Cit3 and Cit4 doublet. Floating point locations for the individual citrate (Cit3 and Cit4) peaks can be determined by calculating the root mean square deviation (RMSD) between the observed citrate peak and a Lorentzian function centered in 0.01 data point increments around the integer peak location. Given the identified Cit3 floating point location, the predicted coupling between the Cit2 and Cit3 peaks can be calculated based on the differences between the Cit3 and TSP locations:

Citr23_coupling=(p1*NMRpH\hat{ }3+p2*NMRpH\hat{ }2+p3*NMRpH+p4)/(NMRpH\hat{ }3+q1*NMRpH\hat{ }2+q2*NMRpH+q3)

where: NMRpH=tsp·fPosition−citr3·fPosition−3700;p1=665.5;p2=−3.613e+05;

p3=8.034e+07;p4=1.251e+09;q1=−680.6;q2=4.383e+05;q3=1.074e+07.

If the value of NMR-pH in the above equation is >300, the predicted coupling between the Cit2 and Cit3 can be calculated based on the difference between the TSP and formate locations:

Citr23_coupling=(p1*NMRpH\hat{ }3+p2*NMRpH\hat{ }2+p3*NMRpH+p4)/(NMRpH\hat{ }3+q1*NMRpH\hat{ }2+q2*NMRpH+q3)

where: NMRpH=tsp·fPosition−formate·fPosition−12050;p1=48.5;p2=−1110;

p3=−1.203e+07;p4=2.466e+09;q1=−455.4;q2=1899;q3=1.383e+07.

The predicted location of the Cit2 peak can be calculated as:

Citr2_location=citr3·fPosition−Citr23_coupling.

In some embodiments, the predicted Cit2 location can be used to define the downfield citrate doublet search region. The downfield citrate doublet search region can be defined as 97 data points downfield from the predicted Citr2 location to 40 data points upfield from the predicted Citr2 location. Within this search region, pairs of peaks matching the known coupling of the Cit1 and Cit2 peaks can be identified. An empirically derived cost function can be then used to choose the pair of peaks of highest amplitude most closely matching the known amplitude relationship of the Cit1 and Cit2 peaks.

The downfield citrate doublet search region can be smoothed using a Savitzky-Golay filter of polynomial order 3 and frame length 9.

The derivative of the smoothed search region can be calculated. Peaks occur where the derivative changes sign from positive to negative.

In some embodiments, for each peak identified, the estimated amplitude of the peak can be calculated by summing the like-signed values of the derivative on either side of the peak location and dividing the sum by 2. In addition, for each peak identified, if a peak paired by 57+6 data points is not already identified as a peak by derivative sign change, the amplitude of the search region paired by 57 data points can be added as “virtual” peak with amplitude calculated as a one-sided sum of like-signed derivate values at the paired point. The addition of “virtual” peaks allows the doublet to be identified even when obscured by other non-citrate peaks. Pairs of peaks (actual and virtual) separated by 57±6 data points are identified. The pair of peaks which maximize the following function are chosen as the downfield citrate doublet (Cit1 and Cit2 peaks):

((((lamp+ramp)/2)\hat{ }2)/abs(21/36−lamp/ramp))/(abs(ref lamp−lamp)+abs(ref ramp−ramp))

where: lamp=estimated amplitude of left (downfield, Cit1) peak;
ramp=estimated amplitude of right (upfield, Cit2) peak;
ref lamp=amplitude of the chosen Cit4 peak;
ref ramp=amplitude of the chosen Cit3 peak.

This empirically derived function can effectively weigh the amplitude relationship of the peak pairs to prefer peak pairs which match the typical amplitude ratio of the Cit1 and Cit2 doublet while also matching the amplitudes of the peaks previously chosen as Cit4 and Cit3, respectively.

In certain embodiments, floating point locations for the individual citrate (Citr1 and Citr2) peaks can be determined by calculating the root mean square deviation (RMSD) between the observed citrate peak and a Lorentzian function centered in 0.01 data point increments around the integer peak location.

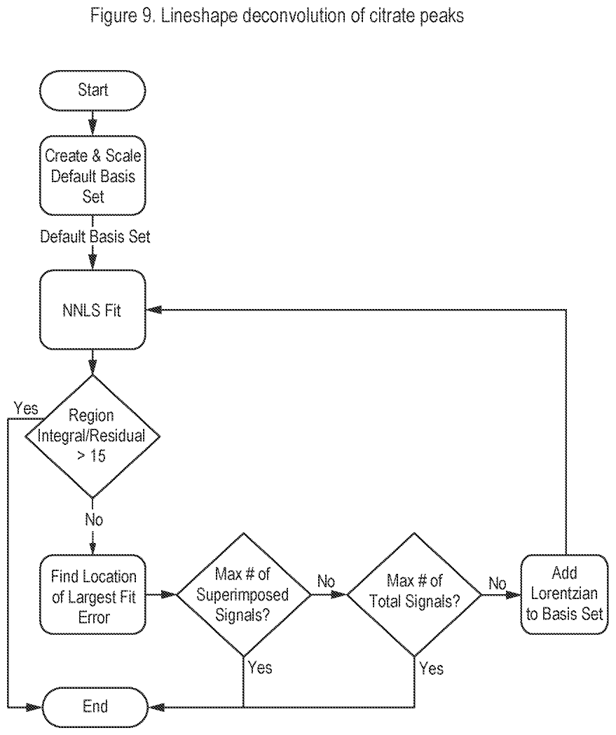

FIG. 9 shows a flow diagram of an embodiment for lineshape deconvolution of citrate NMR peaks. A spectrum from a sample may be evaluated and deconvolved via the flow diagram for lineshape deconvolution. After acquisition, a basis set or design matrix may be generated as a sum of four Lorentzian lineshapes centered on a floating point of the analyte's respective location on peaks. A Lawson-Hanson non-negative least squares algorithm may be used to deconvolve the citrate fitting region.

In some embodiments, the final lineshape attributes of the upfield creatinine signal modeling (CRE1, left and right line widths) may be applied to modeling of the citrate signals. The citrate fitting region may be 50 data points downfield from the integer Cit1 position through 50 data points upfield from the integer Cit4 location.

The citrate components used in the design matrix (basis set) for deconvolution may be constructed as the sum of 4 Lorentzian lineshapes centered on the floating point locations of the 4 citrate peaks. The Lorentzian lineshapes may be divided into left and right line widths.

The left and right line widths may be those from the final model for the upfield creatinine signal (CRE1) adjusted for the nominal relationship between creatinine and citrate line width:

citrllw=1.1643*crellw+0.2347;

citrrlw=1.1643*crerlw+0.2347.

where citrllw and citrrlw may be the left and right citrate line widths respectively, and crellw and crerlw may be the creatinine left and right citrate line widths respectively. The area of the citrate component can be normalized to a value of 1000. By normalizing to a constant area, increases or decreases in the measured area of the citrate signal due to NMR shimming (line width) may be effectively eliminated.

In some embodiments, the design matrix for deconvolution can included 5 citrate components (one centered on the floating point citrate location plus 2 shifted by 2 data points on either side), 2 sloped lines, and a DC offset. A Lawson-Hanson non-negative least squares algorithm may be used to deconvolve the citrate fitting region with this design matrix.

The Lawson-Hanson deconvolution may be performed iteratively. For each iteration after the initial deconvolution, the design matrix is appended with an additional Lorentzian signal positioned at the location of maximum fit deviation. Additional Lorentzians, up to a maximum of 20, may be added to the basis set until the ratio of the fit deviation to area of the citrate fitting region may meet the desired fit quality criteria. If the fitting region area divided by the fit deviation is >15 the fit may be complete. A maximum of 2 additional Lorentzians may be allowed to be superimposed with the citrate peaks as defined by occurring within ±9 data points of any of the 4 citrate peak locations. If location of maximum fit deviation is within ±9 data points of any of the 4 citrate peak locations and 2 additional, superimposed Lorentzians already exist in the design matrix, the fit may be complete. If the maximum of 20 additional Lorentzians are added to the design matrix, the fit may be complete.

From the completed fit, the citrate result (mg/L) can be calculated as the sum of the citrate component deconvolution coefficients*5727*0.1921. The number 5727 can convert the value to μmol/L and the number 0.1921 can convert the μmol/L value to mg/L which may be conventional units for a urine citrate assay.

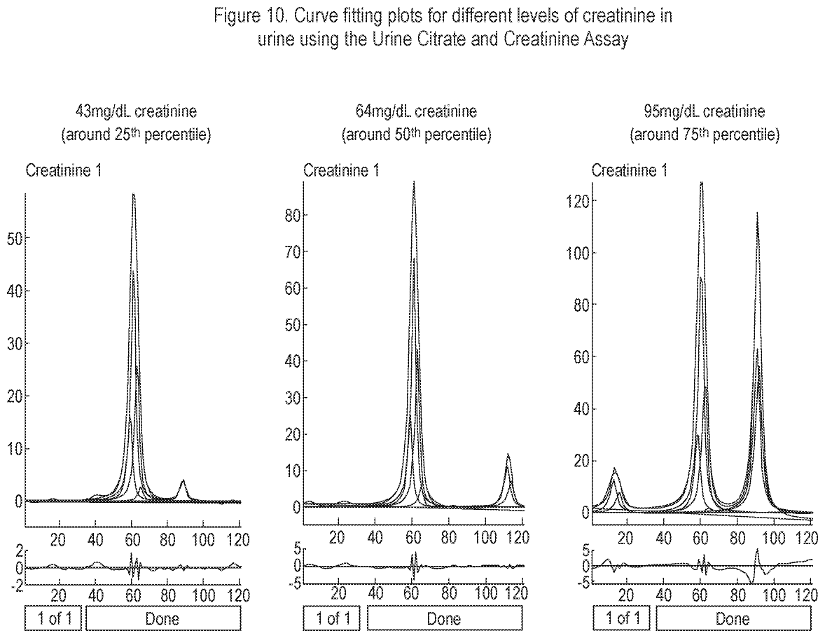

FIG. 10 shows a schematic representation of NMR spectra representative of various concentrations of creatinine according to an embodiment of the disclosure. Further, FIG. 10 displays three mathematically assisted, curve fitting plots corresponding to three different concentrations of creatinine and its respective percentile. In some embodiments, data such as that shown in FIG. 10 may be used for visualizing and determining a concentration of creatinine using its amplitude from a sample.

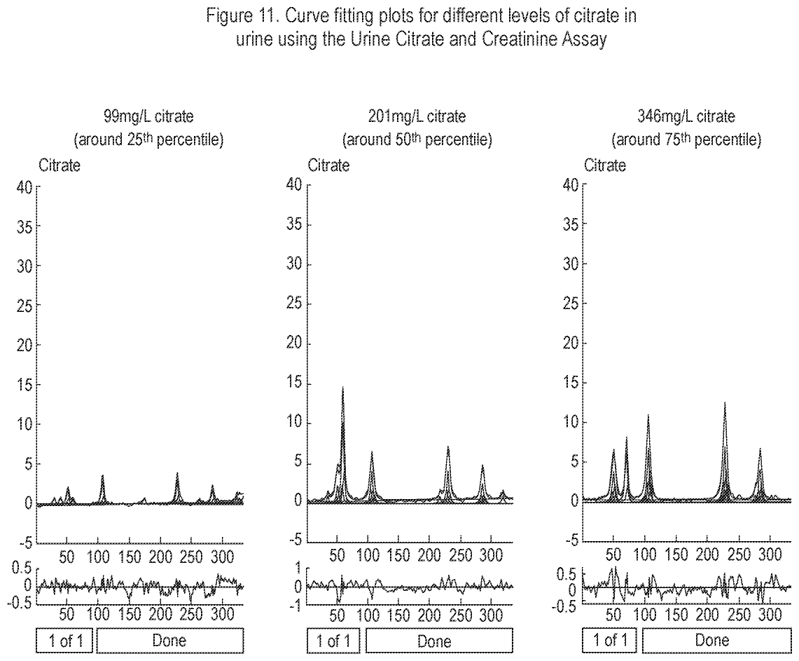

FIG. 11 shows a schematic representation of NMR spectra representative of various concentrations of citrate according to an embodiment of the disclosure. Further, FIG. 11 displays three mathematically assisted, curve fitting plots corresponding to three different concentrations of citrate and its respective percentile. In some embodiments, data such as that shown in FIG. 11 may be used for visualizing and determining a concentration of citrate using its amplitude from a sample.

Systems for Measuring Citrate and/or Creatinine

Also disclosed are systems for performing any of the steps of the disclosed methods and computer-implemented instructions for performing any of the steps of the disclosed methods or running any of the parts of the disclosed systems.

For example, a system may comprise one or more stations or components for performing any of the previous method embodiments. In one embodiment of the present disclosure, a system for determining and measuring a concentration of citrate and/or creatinine may comprise an NMR spectrometer configured to acquire a measured citrate and/or creatinine signal lineshape of an NMR spectrum of a biosample. The NMR analyzer may include a computer program product that may store the measured citrate and/or creatinine lineshapes and reference spectra. The computer program may be configured to derive the concentrations of citrate and/or creatinine through a deconvolution process such as the method steps disclosed herein. The NMR analyzer may further include an NMR spectrometer, a flow probe in communication with the spectrometer, and/or a controller in communication with the spectrometer configured to obtain an NMR signal of a defined peak region of NMR spectra associated with citrate and creatinine in the flow probe.

Also disclosed is a computer-program product tangibly embodied in a non-transitory machine-readable storage medium including instructions configured to cause one or more data processors to perform any of the steps of the disclosed methods or to run any of the components of the disclosed systems. For example, in certain embodiments, disclosed is a computer-program product tangibly embodied in a non-transitory machine-readable storage medium including instructions configured to cause one or more data processors to perform processing comprising: (a) obtaining a sample from a subject; (b) detecting the presence of an analyte(s) of interest in the sample; and (c) calculating the concentration of the analyte(s) of interest in the sample.

In some embodiments, the system may comprise a component to generate a patient report providing citrate and/or creatinine levels.

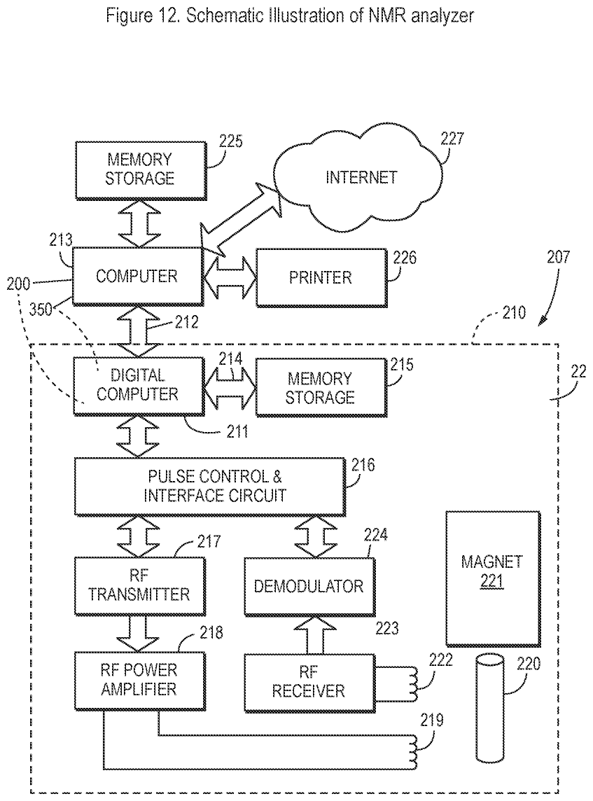

FIG. 12 shows an example of a schematic illustration of an NMR analyzer. A system 207 for acquiring and calculating the lineshape of a selected sample is illustrated. The system 207 may include an NMR spectrometer 22 for taking NMR measurements of a sample. In one embodiment, the spectrometer 22 may be configured so that the NMR measurements are conducted at 400 MHz for proton signals; in other embodiments the measurements may be carried out at between 200 MHz to about 900 MHz or other suitable frequencies. Other frequencies corresponding to a desired operational magnetic field strength may also be employed. Typically, a proton flow probe may be installed, as is a temperature controller to maintain the sample temperature at 47+/−0.5° C. The spectrometer 22 may be controlled by a digital computer 214 or other signal processing unit. The computer 211 may be capable of performing rapid Fourier transformations. It may also include a data link 212 to another processor or computer 213, and a direct-memory-access channel 214 which can connect to a hard memory storage unit 215.

The digital computer 211 may also include a set of analog-to-digital converters, digital-to-analog converters and slow device I/O ports which connect through a pulse control and interface circuit 216 to the operating elements of the spectrometer 22. These elements may include an RF transmitter 217 which may produce an RF excitation pulse of the duration, frequency and magnitude directed by at least one digital signal processor that can be onboard or in communication with the digital computer 211, and/or an RF power amplifier 218 which amplifies the pulse and couples it to the RE transmit coil 219 that surrounds sample cell 220 and/or flow probe 220. The NMR signal produced by the excited sample in the presence of a polarizing magnetic field (e.g., 9.4 Tesla) produced by superconducting magnet 221 may be received by a coil 222 and applied to an RF receiver 223. The amplified and filtered NMR signal may be demodulated at 224 and the resulting quadrature signals may be applied to the interface circuit 216 where they can be digitized and input through the digital computer 211. The circuit 200 and/or module 350, can be located in one or more processors associated with the digital computer 211 and/or in a secondary computer 213 or other computers that may be on-site or remote, accessible via a worldwide network such as the Internet 227.

After the NMR data are acquired from the sample in the measurement cell 220, processing by the computer 211 may be done to produce another file that can, as desired, be stored in the storage 215. This second file may be a digital representation of the chemical shift spectrum that can be subsequently read out to the computer 213 for storage in its storage 225 or a database associated with one or more servers. Under the direction of a program stored in its memory or accessible by the computer 213, the computer 213, which may be a laptop computer, desktop computer, workstation computer, electronic notepad, electronic tablet, smartphone or other device with at least one processor or other computer, may process the chemical shift spectrum in accordance with the teachings of the present disclosure to generate a report which may be output to a printer 226 or electronically stored and relayed to a desired email address or URI. Or, other output devices, such as a computer display screen, electronic notepad, smartphone and the like, may also be employed for the display of results.

The functions performed by the computer 213 and its separate storage 225 may also be incorporated into the functions performed by the spectrometer's digital computer 211. In such case, the printer 226 may be connected directly to the digital computer 211. Other interfaces and output devices may also be employed, as are well-known to those skilled in this art.

Certain embodiments of the present disclosure are directed at providing methods, systems and/or computer program products that use citrate and creatinine evaluations that may be particularly useful in automated screening tests of clinical disease states and/or risk assessment evaluations for screening of in vitro biosamples.

Embodiments of the present disclosure may take the form of an entirely software embodiment or an embodiment combining software and hardware aspects, all generally referred to herein as a “circuit” or “module.”

The present disclosure may be embodied as an apparatus, a method, data or signal processing system, or computer program product. Accordingly, the present disclosure may take the form of an entirely software embodiment, or an embodiment combining software and hardware aspects. Furthermore, certain embodiments of the present disclosure may take the form of a computer program product on a computer-usable storage medium having computer-usable program code means embodied in the medium. Any suitable computer readable medium may be utilized including hard disks, CD-ROMs, optical storage devices, or magnetic storage devices.

The computer-usable or computer-readable medium may be, but is not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium, upon which the program is printed, as the program can be electronically captured, via, for instance, optical scanning of the paper or other medium, then compiled, interpreted, or otherwise processed in a suitable manner if necessary, and then stored in a computer memory.

Computer program code for carrying out operations of the present disclosure may be written in an object-oriented programming language such as java7, Smalltalk, Python, Labview, C++, or VisualBasic. However, the computer program code for carrying out operations of the present disclosure may also be written in conventional procedural programming languages, such as the “C” programming language or even assembly language. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

The flowcharts and block diagrams of certain of the figures herein illustrate the architecture, functionality, and operation of possible implementations of analysis models and evaluation systems and/or programs according to the present disclosure. In this regard, each block in the flow charts or block diagrams represents a module, segment, operation, or portion of code, which comprises one or more executable instructions for implementing the specified logical function(s). It should also be noted that in some alternative implementations, the functions noted in the blocks might occur out of the order noted in the figures. For example, two blocks shown in succession may in fact be executed substantially concurrently or the blocks may sometimes be executed in the reverse order, depending upon the functionality involved.

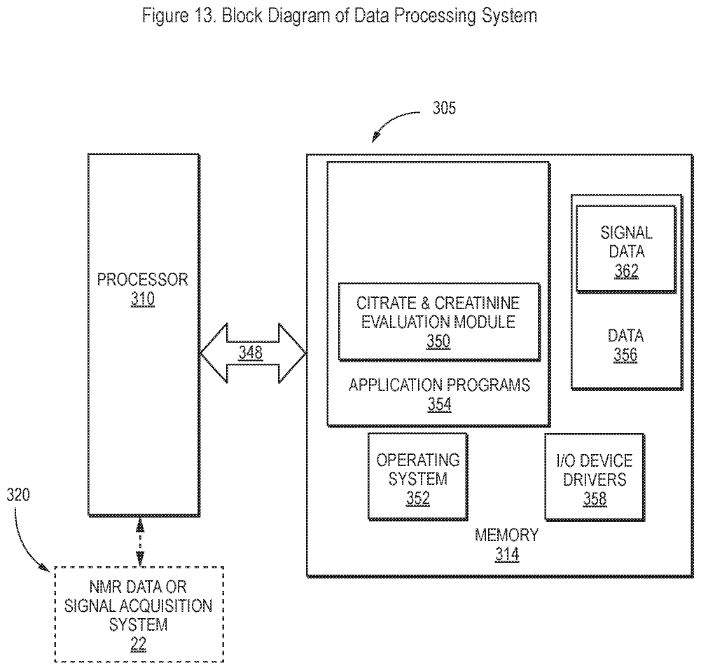

FIG. 13 is a block diagram of exemplary embodiments of data processing systems 305 that illustrates systems, methods, and computer program products in accordance with embodiments of the present disclosure. The processor 310 communicates with the memory 314 via an address/data bus 348. The processor 310 can be any commercially available or custom microprocessor. The memory 314 is representative of the overall hierarchy of memory devices containing the software and data used to implement the functionality of the data processing system 305. The memory 314 can include, but is not limited to, the following types of devices: cache, ROM, PROM, EPROM, EEPROM, flash memory, SRAM, and DRAM.

As shown in FIG. 13, the memory, 314 may include several categories of software and data used in the data processing system 305: the operating system 352; the application programs 354; the input/output (I/O) device drivers 358; a citrate and creatinine Evaluation Module 350; and the data 356. The citrate and creatinine Evaluation Module 350 can deconvolve NMR signal to reveal a defined NMR signal peak region in proton NMR spectra of a respective biosample to identify a level of citrate and/or creatinine.

The data 356 may include signal (constituent and/or composite spectrum lineshape) data 362 which may be obtained from a data or signal acquisition system 320 (e.g., NMR spectrometer 22 and/or analyzer 22). As will be appreciated by those of skill in the art, the operating system 352 may be any operating system suitable for use with a data processing system, such as OS/2, AIX or OS/390 from International Business Machines Corporation, Armonk, N.Y., WindowsCE, WindowsNT, Windows95, Windows98, Windows2000, WindowsXP, Windows 10 from Microsoft Corporation, Redmond, Wash., PalmOS from Palm, Inc., MacOS from Apple Computer, UNIX, FreeBSD, or Linux, proprietary operating systems or dedicated operating systems, for example, for embedded data processing systems.

The I/O device drivers 358 typically include software routines accessed through the operating system 352 by the application programs 354 to communicate with devices such as I/O data port(s), data storage 356 and certain memory 314 components and the signal acquisition system 320. The application programs 354 are illustrative of the programs that implement the various features of the data processing system 305 and can include at least one application, which supports operations according to embodiments of the present disclosure. Finally, the data 356 represents the static and dynamic data used by the application programs 354, the operating system 352, the I/O device drivers 358, and other software programs that may reside in the memory 314.

While the present disclosure is illustrated, for example, with reference to the Module 350 being an application program in FIG. 13, as will be appreciated by those of skill in the art, other configurations may also be utilized while still benefiting from the teachings of the present disclosure. Thus, the present disclosure should not be construed as limited to the configuration of FIG. 13, which is intended to encompass any configuration capable of carrying out the operations described herein.

In certain embodiments, the Module 350 includes computer program code for providing a level of citrate and creatinine which may be used as a marker to assess risk of kidney stone formation and/or to indicate whether personalized therapy intervention is desired and/or track efficacy of a therapy or even an unintended consequence of a therapy.

### EXAMPLES

A method for examining risk of kidney stone formation is described herein. The Urine Citrate and Creatinine Assay (UCC) analyzes a urine biosample in vitro. The process of measuring citrate and creatinine involves sample obtainment from a subject, sample preparation, acquisition of a proton NMR spectrum from sample, determination of citrate and creatinine concentrations using peak(s) residing at 2.50-2.75 ppm, and at approximately 3.07 and 4.12 ppm, respectively, deconvolution of peaks using an algorithm which can identify peaks in a spectrum sample over other non-relevant peaks, and finally, concentration output, using respective peak amplitudes, calculated and produced from the analyzer, such as a Vantera® Clinical Analyzer, following analysis of the sample.

The comparison of the results from the NMR-based assays for urine citrate and creatinine to a chemical-based assay, revealed high correlation coefficients (0.98 and 0.96, respectively), small intercepts (4.7 and 0.97, respectively) and slopes of 0.971 and 0.968, respectively, suggesting that the NMR-based results can substitute for the chemistry-based results. Precision studies showed that the NMR-based assays had good precision (% CV for both assays <3.7%) and measured citrate and creatinine accurately. Finally, while urine preservatives such as acetic acid and boric acid are listed as limitations for the chemistry-based assays, this interference was not found to be a limitation for the NMR-based assay. Therefore, the NMR-based assay has performance characteristics that would allow it to be used for clinical decision-making purposes.

Besides having good performance characteristics for quantifying citrate and creatinine, the NMR-based assay has several benefits over the chemistry-based assays. Some of these benefits are not having the same limitations as the chemistry assays which have the limitation of not being able to test samples where acetic acid or boric acid have been used as preservatives. The NMR assay is reagentless and therefore not dependent on reagents such as citrate lyase that can be affected by supply chain issues. Moreover, this NMR assay is high-throughput and reagentless, there is no manipulation of the sample before testing (e.g., dilution of the sample with diluent buffer occurs on board the instrument), and the turn-around-time for testing and reporting of results is <2 min. Furthermore, the NMR-based assay provides results for both citrate and creatinine simultaneously from the same spectral acquisition for the same specimen. One of the benefits of NMR is that several analytes can be quantified simultaneously which significantly reduces time and resources, as well as the costs for testing. The fact that NMR assays are high throughput and easy to use makes them amenable for use in testing samples from large observational and interventional clinical studies. While the current NMR assay can quantify citrate and creatinine in urine specimens, future application of the technology may also include quantification of cystine and uric acid which would allow for a more extensive analysis of risk of kidney stone formation. The newly developed high-throughput NMR assay exhibited good performance and generates results comparable to the currently utilized chemistry tests and provides an alternative means to simultaneously quantify urine citrate and creatinine for clinical and research use.

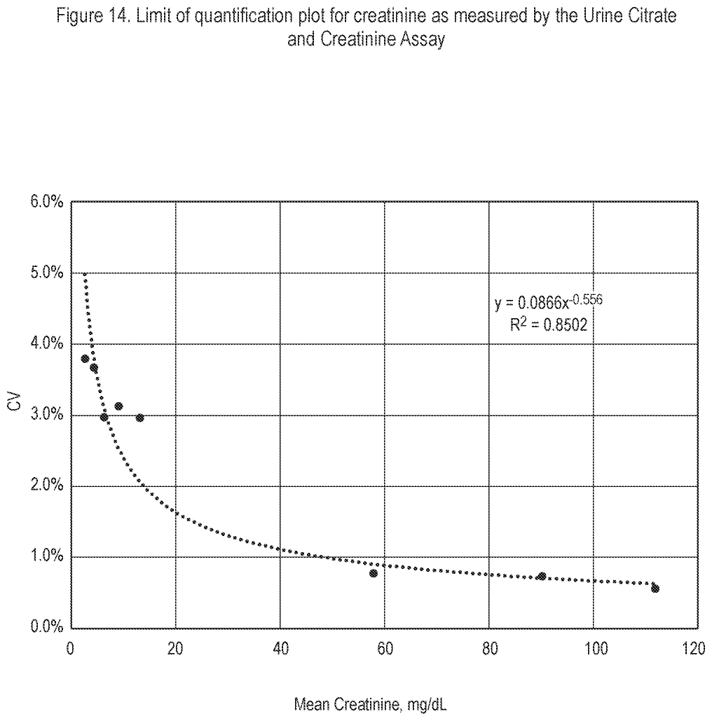

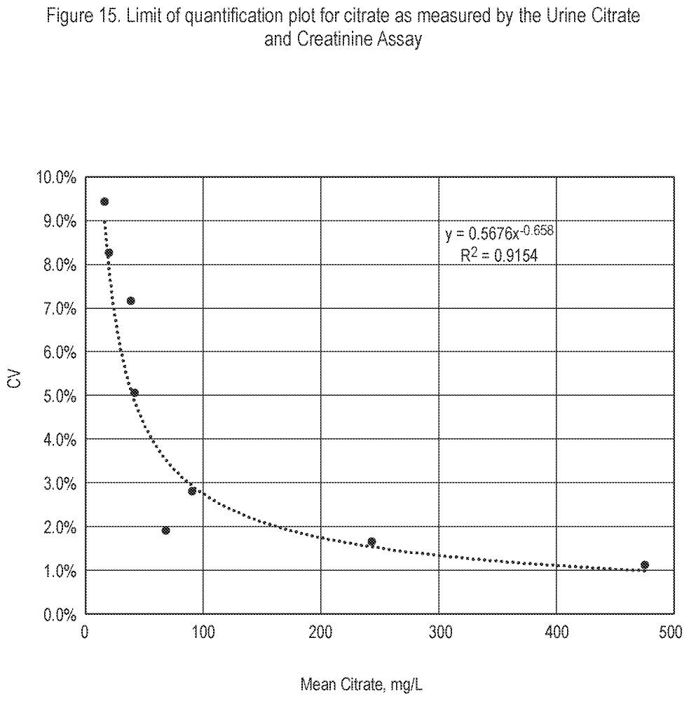

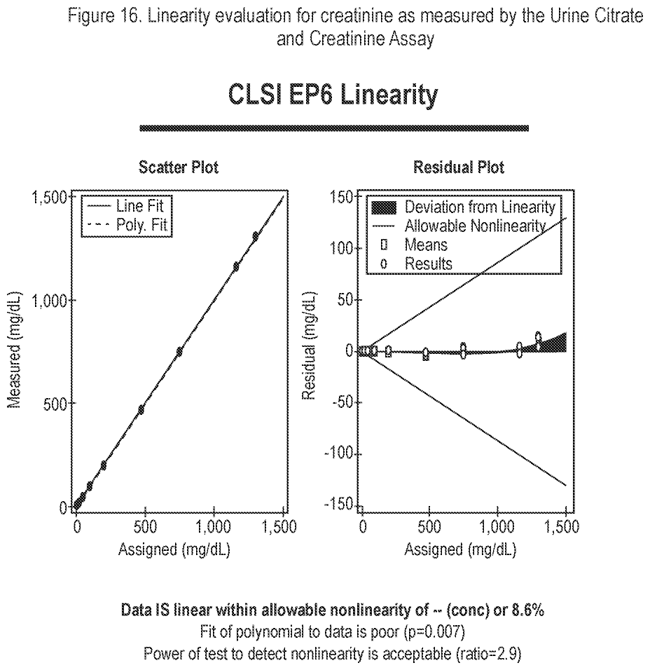

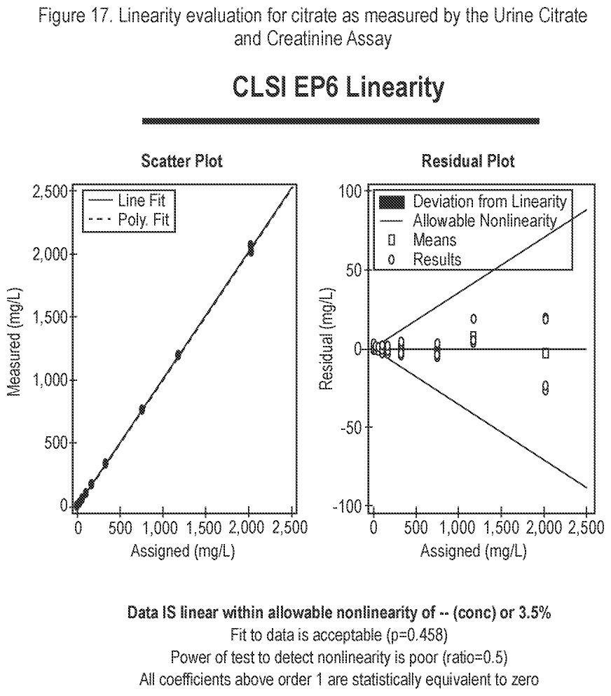

FIGS. 14 and 15 show plots displaying a limit of quantification for creatinine and citrate, respectively. The limit of quantification of the assay was 5.9 mg/dL for creatinine and 17 mg/L for citrate. For the limit of quantification (LOQ), eight urine samples were used to determine the LOQ. Four (4) replicates per pool per day were tested for 3 days according to guidelines set forth by the Clinical and Laboratory Standards Institute (CLSI). The bias limits for citrate and creatinine were predetermined to be 10% and 12.9%, respectively. To calculate the limit of blank (LOB) and limit of detection (LOD), five deionized water samples and five low concentration samples were tested, respectively. Linearity is demonstrated well beyond the reference interval as shown in FIGS. 16 and 17. Creatinine and citrate in urine as measured by the NMR UCC assay compared well with results obtained on a Chemistry analyzer.

FIGS. 16 and 17 show two plots where a first plot (left) is a scatter plot of linearity, and a second plot (right) is a residual plot for creatinine and citrate respectively. Linearity of the assay results was assessed using regression analyses of the assigned versus measured urine citrate and creatinine concentrations. The citrate results were linear over a range of 6 to 2,040 mg/L. The equation for the best line for citrate was determined to be Y=1.01X−0.18. No polynomial fit was statistically better than the linear fit at the 5% significance level. For citrate, the limit of blank (LOB), analytical sensitivity or limit of detection (LOD), and functional sensitivity or limit of quantitation (LOQ) were determined to be: 5, 9 and 17 mg/L, respectively. The creatinine results were linear over a range of 2.8 to 1,308 mg/dL. The equation for the best line for creatinine was determined to be Y=1.00X−0.24. The 3^{rd} order polynomial fit was statistically better than the linear fit at the 5% significance level. For creatinine, the LOB, LOD, and LOQ were determined to be: 5.5, 5.9 and 5.9 mg/dL, respectively.

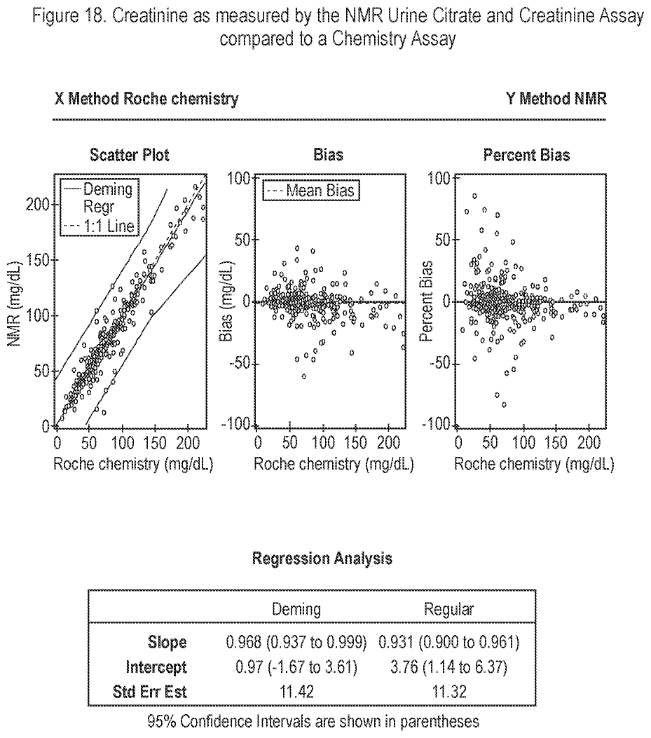

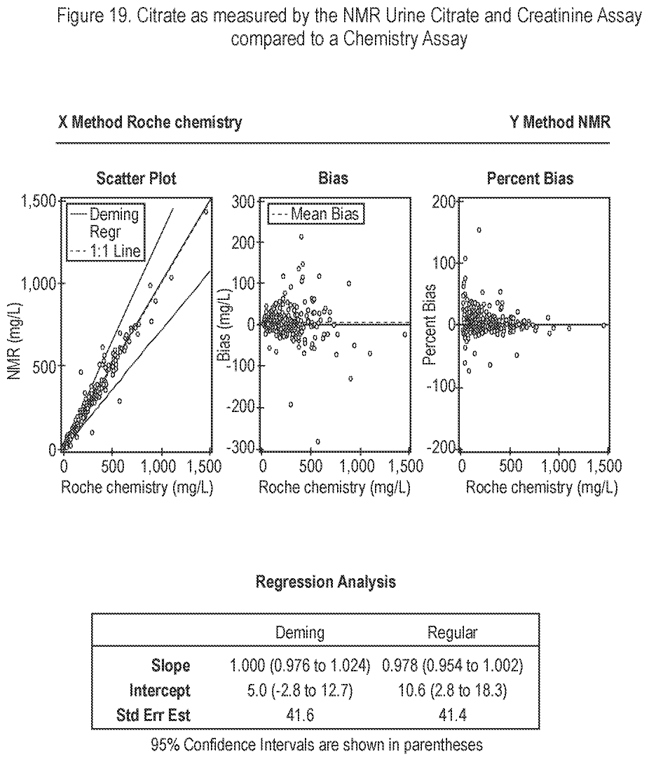

FIGS. 18 and 19 show various plots comparing the measurements of creatinine and citrate made by an NMR assay as compared to a chemistry assay. A method comparison study was performed to compare NMR-based citrate test results with results generated using the chemistry-based assay. Deming regression analysis of the citrate results (n=297) from both assays produced a correlation coefficient of 0.977, and a slope and intercept of 0.971 and 4.7, respectively. The Bias plot revealed no systematic bias between the results of the two assays (mean bias=−3.0%). For creatinine, the NMR-based test results were compared to those generated using the chemistry-based assay. Deming regression analysis of the creatinine results (n=306) from both assays produced a correlation coefficient of 0.960, and a slope and intercept of 0.968 and 0.97, respectively. The Bias plot revealed no systematic bias between the results of the two assays (mean bias=−1.4%).

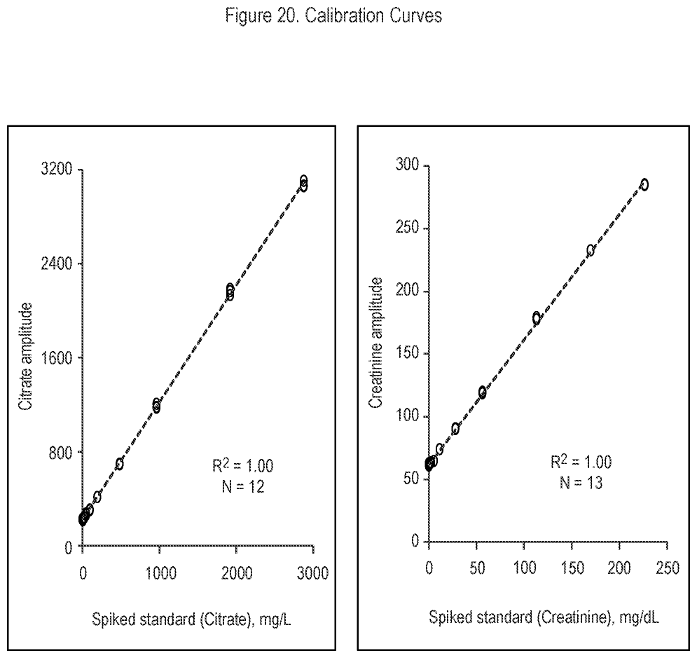

FIG. 20 shows two calibration curves of citrate (left) and creatinine (right) used in conversion from amplitude to concentration. The analyte's signal amplitudes from the deconvolution process were converted to concentration units using a factor obtained from a calibration curve. The calibration curve was generated by relating the peak amplitude for urine spiked with creatinine or citrate standards and the amount of added standards. A urine sample was spiked with creatinine and citrate standards. A total of 12-13 samples with known amounts of added creatinine or citrate were tested in triplicate in order to establish standard curves for creatinine and citrate. The standard curves were used to convert creatinine and citrate from signal amplitudes to concentration units.

Substances (n=10) were tested in vitro for potential interference with results produced by the urine citrate and creatinine assay (3 endogenous and 7 exogenous substances). Pooled urine with citrate concentrations between 228.6-885.7 mg/L and creatinine concentrations between 57.1-128.6 mg/dL were used to generate the substance interference data during the initial screening. Substances that showed interference during the initial screening were tested in a dose response fashion according to CLSI guidelines. For acetic acid and boric acid, which can be used as preservatives in urine, the recommendation is to test 5 times the suggested concentration. For acetic acid, the recommended concentration of 0.5% to 2.5% was tested and for boric acid the recommended concentration of 1% to 5% was tested. The highest concentration tested where there was no interference with citrate and creatinine results was defined as <10% bias for citrate and <12.9% bias for creatinine. Table 3 shows the highest substance concentrations tested that did not elicit interference.

TABLE 3
Results of interference testing showing highest concentration
of substance tested that did not interfere with
citrate or creatinine assay results.

| | | Concentration |
|---|---|---|
| Substance | Drug name | (mg/dL) |
| Urea | — | 263.9 |
| Uric acid | — | 23.5 |
| Protein (albumin) | — | 175.0 |
| Acetaminophen | Tylenol | 21.8 |
| Acetic acid | Preservative | 2500 |
| Acetylsalicylic acid | Aspirin | 66.4 |
| Ascorbic acid | Vitamin C | 6.0 |
| Boric acid | Preservative | 1250* |
| Ibuprofen sodium salt | Advil | 59.0 |
| Naproxen sodium | Aleve | 56.1 |

*Interferes with assay results at this concentration, which is higher than the highest concentration of boric acid when used as a preservative.

### Illustrative Embodiments of Suitable Methods, Systems, and Programs

As used below, any reference to methods, systems, and programs is understood as a reference to each of those methods, systems, and programs disjunctively (e.g., “Illustrative embodiment 1-4 is understood as illustrative embodiment 1, 2, 3, or 4.”).

Illustrative embodiment 1 is a method comprising: acquiring an NMR spectrum of a biosample obtained from a subject; and measuring a concentration of citrate and/or creatinine from the biosample based on the NMR spectrum.

Illustrative embodiment 2 is the method of any preceding or subsequent illustrative embodiment, wherein acquiring the NMR spectrum comprises: producing a measured citrate and/or creatinine signal lineshape from the NMR spectrum; and generating a calculated lineshape for citrate and/or creatinine, wherein the calculated lineshape is based on derived concentrations of citrate and/or creatinine expected in the biosample.

Illustrative embodiment 3 is the method of any preceding or subsequent illustrative embodiment, wherein generating a calculated lineshape for citrate and/or creatinine comprises calculating a plurality of reference coefficients for the calculated lineshape based on a linear least squares fit technique.

Illustrative embodiment 4 is the method of any preceding or subsequent illustrative embodiment, further comprising: determining a degree of correlation between an initial calculated lineshape of the biosample and the measured citrate and/or creatinine signal lineshape of the biosample; and determining a presence of citrate and/or creatinine based on the calculated lineshape if the degree of correlation between the calculated lineshape and the measured citrate and/or creatinine signal lineshape of the biosample is above a predetermined threshold.

Illustrative embodiment 5 is the method of any preceding or subsequent illustrative embodiment, wherein the NMR spectrum of the biosample includes four citrate proton singlet signals in four distinct regions, wherein the four citrate proton singlet signals region comprise a range of 2.50-2.75 ppm.

Illustrative embodiment 6 is the method of any preceding or subsequent illustrative embodiment, wherein the NMR spectrum of the biosample includes two creatinine proton singlet signals in two distinct regions, wherein the two creatinine proton singlet signals region comprise a range of 3.0-4.20 ppm.

Illustrative embodiment 7 is the method of any preceding or subsequent illustrative embodiment, further comprising: deconvolving signal data associated with citrate and/or creatinine proton singlet signals; and comparing data from a plurality of deconvolved signal data with a priori calibration data corresponding to standard biosamples with known concentrations of citrate and/or creatinine to determine the concentration of citrate and/or creatinine in the biosample.

Illustrative embodiment 8 is the method of any preceding or subsequent illustrative embodiment, further comprising a step of producing a report, listing the concentration of citrate and/or creatinine constituents present in the biosample.

Illustrative embodiment 9 is the method of any preceding or subsequent illustrative embodiment, wherein the biosample comprises blood, serum, plasma, sputum, cerebral spinal fluid, urine, or combinations thereof.

Illustrative embodiment 10 is the method of any preceding or subsequent illustrative embodiment, further comprising a step of identifying the subject as one who has a condition associated with elevated or reduced abnormal concentrations of citrate and/or creatinine.

Illustrative embodiment 11 is a system comprising: an NMR spectrometer configured to acquire a measured citrate and/or creatinine signal lineshape of an NMR spectrum of a biosample; a computer program product comprising instructions to store the measured citrate and/or creatinine signal lineshape of the biosample; a computer program product comprising instructions for storing reference spectra for each of citrate and/or creatinine; a computer program product comprising instructions to calculate a lineshape based on a plurality of derived concentrations of citrate and/or creatinine from the biosample and a reference spectra; and a computer program product comprising instructions for comparing the measured citrate and/or creatinine signal lineshape and the calculated lineshape to determine a degree of correlation between the calculated lineshape and the measured citrate and/or creatinine signal lineshape.

Illustrative embodiment 11 is the system of any preceding or subsequent illustrative embodiment, further comprising an output device for producing a report indicating a presence of citrate and/or creatinine.

Illustrative embodiment 12 is the system of any preceding or subsequent illustrative embodiment, configured to carry out the method of any one of illustrative embodiments 1-10.

Illustrative embodiment 13 is a computer program product tangibly embodied in a non-transitory machine-readable storage medium including instructions configured to cause one or more data processors to perform processing comprising a non-transitory machine-readable storage medium including instructions configured to cause one or more data processors to perform processing comprising: obtaining a sample from a subject; detecting the presence of analytes of interest in the sample; and calculating a concentration of the analytes of interest in the sample.

What is claimed is:

1. A method comprising:
acquiring an NMR spectrum of a biosample obtained from a subject; and
measuring a concentration of citrate and/or creatinine from the biosample based on the NMR spectrum.

2. The method of claim 1, wherein acquiring the NMR spectrum comprises:
producing a measured citrate and/or creatinine signal lineshape from the NMR spectrum; and
generating a calculated lineshape for citrate and/or creatinine,
wherein the calculated lineshape is based on derived concentrations of citrate and/or creatinine expected in the biosample.

3. The method of claim 2, wherein generating a calculated lineshape for citrate and/or creatinine comprises calculating a plurality of reference coefficients for the calculated lineshape based on a linear least squares fit technique.

3. The method of claim 1, wherein the NMR spectrum of the biosample includes four citrate proton singlet signals in four distinct regions, wherein the four citrate proton singlet signals region comprise a range of 2.50-2.75 ppm.

4. The method of claim 2 further comprising:
determining a degree of correlation between an initial calculated lineshape of the biosample and the measured citrate and/or creatinine signal lineshape of the biosample; and
determining a presence of citrate and/or creatinine based on the calculated lineshape if the degree of correlation between the calculated lineshape and the measured citrate and/or creatinine signal lineshape of the biosample is above a predetermined threshold.

4. The method of claim 1, wherein the NMR spectrum of the biosample includes two creatinine proton singlet signals in two distinct regions, wherein the two creatinine proton singlet signals region comprise a range of 3.0-4.20 ppm.

7. The method of claim 1, further comprising:
deconvolving signal data associated with citrate and/or creatinine proton singlet signals; and
comparing data from a plurality of deconvolved signal data with a priori calibration data corresponding to standard biosamples with known concentrations of citrate and/or creatinine to determine the concentration of citrate and/or creatinine in the biosample.

8. The method of claim 7, further comprising a step of producing a report, listing the concentration of citrate and/or creatinine constituents present in the biosample.

9. The method of claim 1, wherein the biosample comprises blood, serum, plasma, sputum, cerebral spinal fluid, urine, or combinations thereof.

10. The method of claim 1, further comprising a step of identifying the subject as one who has a condition associated with elevated or reduced abnormal concentrations of citrate and/or creatinine.

11. A system comprising:
an NMR spectrometer configured to acquire a measured citrate and/or creatinine signal lineshape of an NMR spectrum of a biosample;
a computer program product comprising instructions to store the measured citrate and/or creatinine signal lineshape of the biosample;
a computer program product comprising instructions for storing reference spectra for each of citrate and/or creatinine;
a computer program product comprising instructions to calculate a lineshape based on a plurality of derived concentrations of citrate and/or creatinine from the biosample and a reference spectra; and
a computer program product comprising instructions for comparing the measured citrate and/or creatinine signal lineshape and the calculated lineshape to determine a degree of correlation between the calculated lineshape and the measured citrate and/or creatinine signal lineshape.

12. The system of claim 11, further comprising an output device for producing a report indicating a presence of citrate and/or creatinine.

13. A computer program product tangibly embodied in a non-transitory machine-readable storage medium including instructions configured to cause one or more data processors to perform processing comprising a non-transitory machine-readable storage medium including instructions configured to cause one or more data processors to perform processing comprising:
obtaining a sample from a subject;
detecting the presence of analytes of interest in the sample; and
calculating a concentration of the analytes of interest in the sample.
